(19) United States
(12) Patent Application Publication
(10) Pub. No.: US 2005/0171338 A1
(43) Pub. Date: Aug. 4, 2005

(54) Mammalian tribbles signaling pathways and methods and reagents related thereto

(76) Inventors: Steven Dower, Sheffield (GB); Eva Quanstrom, Sheffield (GB); Endre Kiss-Toth, Sheffield (GB)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/466,020

(22) PCT Filed: Jan. 8, 2002
(86) PCT No.: PCT/US02/00070

Related U.S. Application Data
(60) Provisional application No. 60/260,294, filed on Jan. 8, 2001.

(51) Int. Cl. C12Q 1/68; C07H 21/04; C12P 21/02; C07K 14/54

(52) U.S. Cl. 530/351; 435/6; 435/69.5; 435/320.1; 435/325; 536/23.5

(57) ABSTRACT

The invention provides methods and reagents for modulating mitogen activated protein kinase pathways using mammalian tribbles homologs (htrb).

### 1. BACKGROUND OF THE INVENTION

The function of immune and inflammatory genes play a central role in the pathology of many diseases including rheumatoid arthritis, inflammatory bowel disorder, psoriasis, and Alzheimer's disease. There is evidence to suggest that these immune and inflammatory genes function as a complex network of interdependent signaling components. These signaling components mediate signaling events which take place both extracellularly (e.g. through the action of various cytokines such as the interleukins) and intracellulary (e.g. through the action of signal transducing kinases and transcriptional regulators such as AP-1 and NF-B). A variety of means exist for regulating inflammatory responses involved in disease processes. For example, aspirin (salicylic acid) inhibit activation of NF-kB by blocking I-kB kinase, a key enzyme in NF-kB activation. Sulfasalazine and gold compounds also inhibit NF-kB activation. Glucocorticoids suppress expression of inflammatory genes by binding glucocorticoid receptors involved in NF-kB activation. Such drugs are commonly used to regulate inflammatory diseases such as rheumatoid arthritis. Nevertheless there is a need to develop drugs with particular anti-inflammatory specificities that are particularly adapted to controlling certain aberrant inflammatory processes while allowing nonpathological inflammatory processes to continue without interference. Indeed, a variety of anti-inflammatory medicaments would benefit the development of optimized drug treatments for specific patients with particular needs (see Davies & Skjodt (2000) Clin Pharmoacokinet 38: 377-92). Furthermore, continuing advances in understanding the molecular mechanisms of inflammation will benefit the development of more effective, more specific and less toxic drugs to control inflammatory diseases. An understanding of the relationships between the genes comprising this network would provide a broad array of drug targets for the control of autoimmune and inflammatory disease processes. Molecular agonists and antagonists could be designed to act alone or in concert at one or more points in this gene network in order to effect control of the disease process.

A large body of work has recently been focused on signalling networks triggered by proinflammatory cytokines, bacterial cell walls and shear stress. In general, two major signalling cascades are activated by these stimuli. The major intracellular signaling cascades involved in immune and inflammatory gene network regulation are the mitogen activated protein kinase (MAPK, or stress kinase) cascade and the IkB kinase cascade as well as the JAK/STAT signal transduction pathway (see e.g. Rivest et al. (2000) Proc Soc Exp Biol Med 223: 22-38). Activation of NF-kB is thought to be mediated primarily via I-kB kinase (IKK), whereas that of AP-1/ATF can be mediated by stress-activated protein kinases (SAPKs; also termed Jun kinases or JNKs). IKKalpha and IKKbeta are two catalytic subunits of a core IKK complex that also contains the regulatory subunit NEMO (NF-kappaB essential modulator)/IKKgamma. The latter protein is essential for activation of the IKKs, but its mechanism of action is not known, although the molecular cloning of CIKS (connection to IKK and SAPK/JNK), a previously unknown protein that directly interacts with NEMO/IKKgamma in cells, may prove informative (see Leonardi et al. (2000) PNAS, USA 97: 10494-9). When ectopically expressed, CIKS stimulates IKK and SAPK/JNK kinases and it transactivates an NF-kappaB-dependent reporter. Activation of NF-kappaB is prevented in the presence of kinase-deficient, interfering mutants of the IKKs. CIKS may help to connect upstream signaling events to IKK and SAPK/JNK modules and CIKS could coordinate the activation of two stress-induced signaling pathways, functions reminiscent of those noted for tumor necrosis factor receptor-associated factor adaptor proteins.

Individual stress kinase mediated pathways behave differently in different cell types. Specificity may be achieved in part by cell type specific expression of certain pathway components such as CIKS. It is important that all components contributing to the regulation and specificity of these mitogen activated protein kinase signalling pathways be identified as each represents a target for regulation of this important class of inflammatory signalling events.

### 2. SUMMARY OF THE INVENTION

The invention is based in part upon the cloning and identification of certain mammalian htrbs genes and encoded htrbs proteins which function as inhibitors of particular stress kinase pathways. The htrbs genes are mammalian homologs of the Drosophila tribbles gene, which coordinates mitosis with morphogenesis and cell fate determination in fruit fly development (see Mata et al. (2000) Cell 101: 511-22; and Grosshans & Wieschaus (2000) Cell 101: 523-31). The invention provides a human homolog of the Drosophila tribbles gene which has been termed htrb-1, for human tribbles homologue-1 (also known as homo SKIP1 (Gen Bank Accession NO. AF250310). The htrb-1 inhibits basal but not induced activity of the cytokine responsive interleukin-1 (IL-8) gene reporter, which is responsive to both NF-kB and AP-1 induction through binding sites for these transcriptional activators present in the IL-8 promoter. The htrb-1 gene of the invention specifically represses AP-1 but not NF-kB or JAK/STAT mediated transcriptional induction. Therefore the htrb-1 gene of the invention provides a convenient and a specific tool for modulating stress kinase-induced pathways.

In preferred embodiments, the invention provides an isolated htrb-1 encoding nucleic acid comprising a nucleotide sequence which is at least about 90% identical to the nucleotide sequence set forth in SEQ ID No. 1 or the complement thereof and more preferably at least about 95% or 99% identical. In certain embodiments, the nucleic acid of the invention further an AP-1 activation inhibitory activity. The invention further provides isolated nucleic acid which encodes an htrb polypeptide, such as a polypeptide that is at least about 75% identical to the htrb-1 polypeptide sequence set forth in SEQ ID No. 2, and which, preferably, further encodes an AP-1 inhibitory activity. In certain preferred embodiments, the isolated nucleic acid of the invention encodes an htrb bioactivity such as an IL-8 basal expression inhibitory activity, and AP-1 transcriptional activation inhibitory activity, an MEKK-1 kinase signaling inhibitory activity, an MKK-7 kinase signaling inhibitory activity, an ERK kinase signaling inhibitory activity, a JNK kinase signaling inhibitory activity, or a cellular hypertrophy-promoting activity. Preferred nucleic acids of the invention include isolated nucleic acid with a nucleotide sequence that hybridizes under stringent conditions to certain particular htrb-1 nucleotide sequences such as: nucleotides 1 to 448 of SEQ ID No. 1; nucleotides 1 to 729 of SEQ ID No. 1; and nucleotides 1500 to 1916 of SEQ ID No. 1.

The invention further provides isolated htrb polypeptides which include a polypeptide sequence of at least 10, and, more preferably, 20 or 30 contiguous amino acids from the htrb-1 sequence spanning amino acid residues 1 to 150 of SEQ ID No. 2. Preferably the htrb polypeptides of the invention are at least about 70%, and, more preferably 80, 90 95 or 99% identical to the htrb-1 sequence set forth in SEQ ID No. 2. In preferred embodiments, the htrb polypeptide encodes an htrb-1 bioactivity such as an ability to: inhibit IL-8 basal expression, inhibit AP-1 transcriptional activation, inhibit MEKK-1 kinase signaling, inhibit MKK-7 kinase signaling, inhibit ERK kinase signaling, inhibit JNK kinase signaling, or promote cellular hypertrophy.

In particularly preferred embodiments, the invention provides methods of modulating an AP-1 mediated inflammatory signal in a cell by providing the cell with a htrb agonist or antagonist. The htrb agonist or antagonist can be an htrb polypeptide, an htrb peptidomimetic or an htrb nucleic acid. Preferred htrb nucleic acid agonist or antagonists of the invention include htrb-1, htrb-1 N htrb-1 C, htrb-1 N C, htrb-3, an htrb-1 5′ UTR and N-terminal variable region antisense construct, an htrb-3 5′ UTR and N-terminal variable region antisense construct, and an htrb-1 3′UTR sense construct. Preferred htrb polypeptide agonist or antagonists of the invention include htrb-1, htrb-1 N htrb-1 C, htrb-1 N C, htrb-3, htrb-3 N htrb-3 C, and htrb-3 N C. The method of the invention may be used to inhibit an AP-1 mediated inflammatory signal such as a TNF induced inflammatory signal, or an interleukin induced inflammatory signal. The method of the invention further provides a method of activating an ERK-mediated signal in a cell by providing the cell with an htrb agonist activity. The ERK-mediated signal may be, for example, an AP-1-mediated gene activation signal, an estrogen receptor-mediated gene activation signal, an FGF induced signal, or a PMA induced signal.

A particularly preferred embodiment of the invention provides a method of identifying an interleukin regulatory gene by a particular cloning process. The process of the invention includes: (1) transfecting a mammalian reporter cell comprising an interleukin gene or inflammatory gene reporter with a low-complexity pool of a mammalian cDNA vector library; (2) screening the transfected reporter cell for positive clones by identifying transfected cells with either an increase or decrease in the interleukin gene reporter activity relative to the mammalian reporter cell transfected with the vector alone; and (3) identifying the interleukin regulatory gene from the positive clones by retransfecting the low complexity pool from said positive clones and sequencing the cDNA inserts from the positive clones obtained upon retransfection, so as to identify an interleukin regulatory gene. The interleukin or inflammatory gene reporter may be an IL-1A gene reporter, an IL-1B gene reporter, an IL-1RN gene reporter, or an IL-8 gene reporter. Preferred cells for use in this aspect of the invention include mammalian cells such as HeLa cells, NIH 3T3 cells, Raw cells, or peripheral blood lymphocytes. Preferred libraries for use in this aspect of the invention includes mammalian cDNA libraries such as PBMC libraries, HeLa cell libraries, PMA-induced mammalian cell libraries, or a mammalian cell library constructed from another cytokine-induced mammalian cell such as an IL-5, TGF-beta, interferon-alpha, or IL-12 induced mammalian cell.

In preferred embodiments of this aspect of the invention, an interative doing process is used to derive still other inflammatory regulatory network genes. This process of the invention involves: first expressing an interleukin regulatory gene clone, comprising an interleukin regulatory gene cDNA and an expression vector, in a population of mammalian cells; next isolating a population of nucleic acids representing expressed genes from said cells; determining the gene expression profile of the interleukin regulatory gene expressing cells by microarray analysis of the population of nucleic acids representing expressed genes from said cells; and then comparing the gene expression pattern of mRNA expression from the cells transfected with the interleukin regulatory gene clone with that obtained by transfecting the vector alone in order to identify genes, other than the said interleukin regulatory gene, which are either up-regulated or down-regulated in the interleukin regulatory gene expressing cells, so as to identify the other gene targets of the interleukin regulatory gene making up the inflammatory signaling network. In preferred embodiments the method assesses altered expression of responsive genes by microarray analysis such as gene transcription profiling or gene expression fingerprinting.

### 3. BRIEF DESCRIPTION OF THE FIGURES

FIG. 1 shows htrb-1 mediated repression of basal, but not IL-1 beta- or TNF alpha- activated, expression of IL-8 reporter (panel A); the specific repression of AP-1 activation (panel B) but not NF-kB activation (panel C) by htrb-1; a comparison of htrb-1 and htrb-3 mediated repression of AP -1 (panel E); the repression of MEKK-1 mediated AP-1 activation (squares, horizontal axis) by htrb-1 (triangles); and htrb-3 repression of p38-mediated activation.

FIG. 2 shows an alignment of htrb-1, htrb-3 and related tribbles gene sequences

FIG. 3 shows expression of antisense htrb-1 and htrb-3 RNA inhibits stress kinase activation.

FIG. 4 shows that htrb-1 inhibits MEKK-1 and MKK7 mediated AP-1 activation.

FIG. 5 shows that htrb-1 and htrb-3 alter c-Jun and ERK phosphorylation kinetics.

FIG. 6 shows deletion analysis of htrb-1.

FIG. 7 shows that htrb genes are expressed and act in a tissue-specific manner.

FIG. 8 shows the detection of htrb-1 and htrb-3 expression by confocal microscopy.

FIG. 9 shows the titration of sense vs. antisense htrb-3.

FIG. 10 shows the nucleic acid (panel A) and polypeptide sequence of htrb-1 (GenBank Accession No. AF250310).

FIG. 11 shows the nucleic acid (panel A) and polypeptide sequence of htrb-3 (GenBank Accession No. AF250311).

Table 1 shows that htrb-3 inhibits AP-1, but not NF-kB, induction by a variety of cytokines.

### 4. DETAILED DESCRIPTION OF THE INVENTION

4.1. General

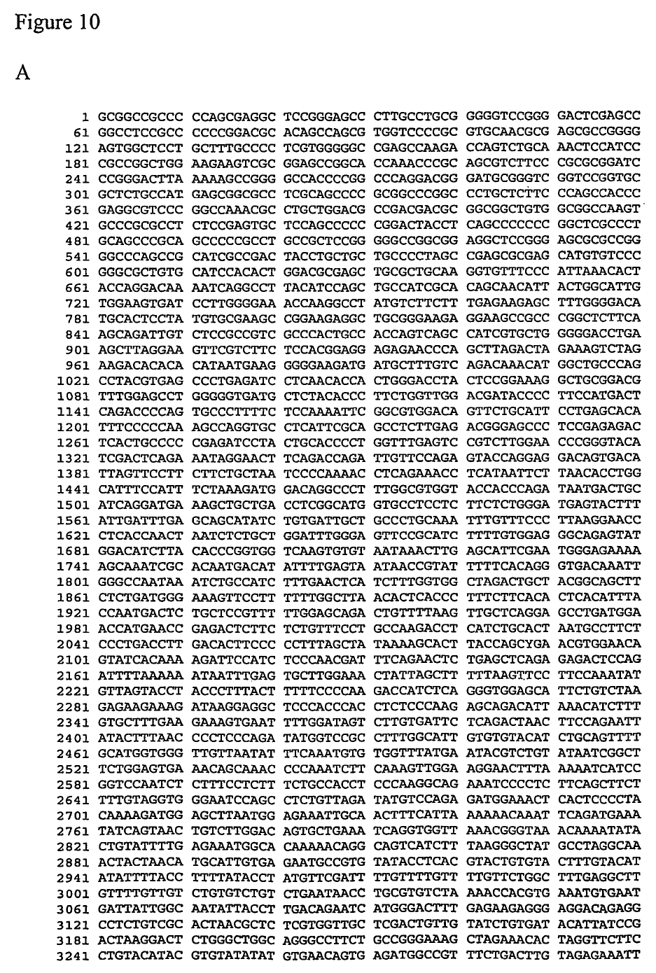

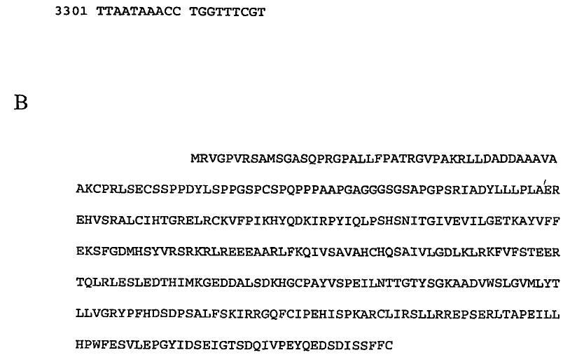

The invention is based in part upon the cloning and identification of certain mammalian htrbs genes and encoded htrbs proteins which function as inhibitors of particular stress kinase pathways. The htrbs genes are mammalian homologs of the Drosophila tribbles gene, which coordinates mitosis with morphogenesis and cell fate determination in fruit fly development (see Mata et al. (2000) Cell 101: 511-22; and Grosshans & Wieschaus (2000) Cell 101: 523-31). The invention provides a human homolog of the Drosophila tribbles gene which has been termed htrb-1, for human tribbles homologue-1. The htrb-1 gene is also known as the human SKIP1 gene and the nucleic acid sequence (SEQ ID NO. 1) and corresponding polypeptide sequence (SEQ ID NO. 2) are described in GenBank Accession NO. AF250310 and shown in FIG. 10. SEQ ID NO. 1 nucleotides 282 to 1400 correspond to the ORF of the htrb-1 gene which encode the htrb-1 polypeptide (SEQ ID NO. 2). The htrb-1 gene inhibits basal but not induced activity of the cytokine responsive interleukin-1 (IL-8) gene reporter, which is responsive to both NF-kB and AP-1 induction through binding sites for these transcriptional activators present in the IL-8 promoter. The htrb-1 gene of the invention specifically represses AP-1 but not NF-kB or JAK/STAT mediated transcriptional induction. Therefore the htrb-1 gene of the invention provides a convenient and a specific tool for modulating stress kinase-induced pathways.

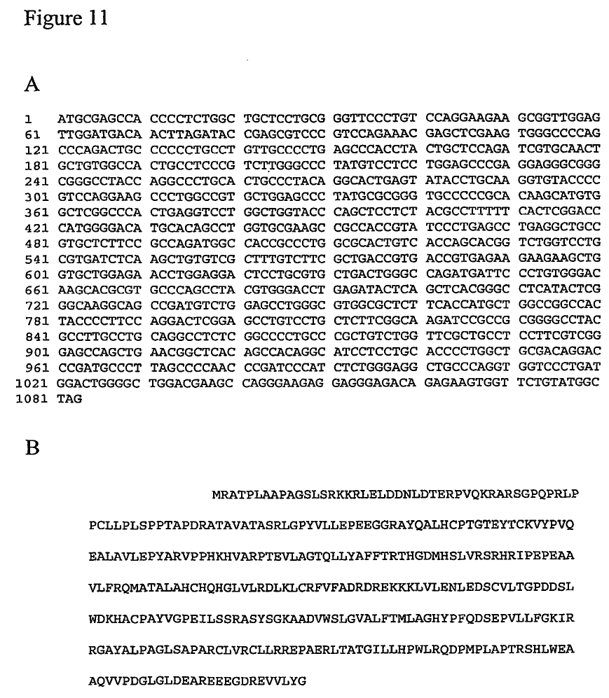

Mitogen Activated Protein Kinase cascades are activated by a wide variety of extracellular stimuli. However, the individual pathways respond differently in different cell types. This specificity is achieved partially by cell type specific expression of certain pathway components. The instant invention provides a family of mammalian tribbles homologs (htrbs) which influence the activity of MAPK pathways. Expression of these proteins is tightly regulated (Mayumi-Matsuda, K. et al. (1999) Biochemical and Biophysical Research Communications 258:260-64; Wilkin, F. et al. (1997) European Journal of Biochemistry 248:660-68). Overexpression of htrbs or suppression of endogenous levels leads to the inhibition of a stress kinase responsive reporter, suggesting that these proteins represent a rate-limiting component of MAPK pathways. The mechanism of htrb action involves control of phosphorylation of extracellular signal regulated kinases (ERKs), which is strongly potentiated by elevated htrb levels. In addition, optimal activation of ERK, JNK and p38 responsive reporters by upstream kinases is achieved in the presence of different amounts of htrb, suggesting that selective activation of individual MAPK pathways occurs by regulation of htrb expression. There is an increasing body of experimental evidence, supported by mathematical models, (Levchenko, A. et al. (2000) Proc. Natl. Acad. Sci. USA 97:5818-23), to suggest, that regulation of the expression levels of scaffolding components is an effective way to limit the amplitude of signalling responses (e.g. Cacace, A.M. et al. (1999) Molecular and Cellular Biology 19:229-40; Yasuda, J. et al. (1999) Mol. Cell. Biol. 19:7245-54). Based on our observations we suggest a model where htrb proteins are scaffolds, regulating the dynamics of MAPK mediated cellular responses. The invention further provides htrb-2 and htrb-3 genes, which are 41% and 51% identical, respectively, to the htrb-1 gene. The htrb-3 gene is also known as the human SKIP3 gene and the nucleic acid sequence (SEQ ID NO. 3) and corresponding polypeptide sequence (SEQ ID NO. 4) are described in GenBank Accession NO. AF250311 and shown in FIG. 11. Nucleotides 1 to 1083 of SEQ ID NO. 3 correspond to the ORF of htrb-3 and, accordingly, encode the htrb-3 polypeptide corresponding to SEQ ID No. 4. The invention still further provides methods of using these compositions to control inflammatory signaling.

The invention further provides methods for the cloning and identification of other genes that are components of an inflammatory signaling network. In preferred embodiments, this method includes screening of low-complexity pools of cDNA for genes which effect an increase or a decrease in the basal or induced levels of an interleukin gene reporter. In certain embodiments, the method of the invention involves expression screening for gene products which modulate the activity of a cytokine responsive reporter construct, such as an interleukin 8 (IL-8) reporter construct. Interleukin (IL)-8 is a C-X-C chemokine that plays an important role in acute inflammation through its G protein-coupled receptors CXCR1 and CXCR2. In particularly preferred embodiments, further components of the gene network are cloned and identified by an iterative process involving micro-array analysis with the interleukin regulatory genes thus identified. The goals of these methods of biotechnological business include the identification of a broad array of drug targets for the control of autoimmune and inflammatory disease processes. A further goal includes the design of molecular agonists and antagonists which may act alone or in concert at any one or more points in the inflammatory gene network to effect control of an autoimmune or inflammatory disease process.

4.2. Definitions

For convenience, the meaning of certain terms and phrases employed in the specification, examples, and appended claims are provided below.

The term “aberrant activity”, as applied to an activity of a polypeptide such as htrb, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant htrb activity due to overexpression or underexpression of the gene encoding htrb.

The term “agonist”, as used herein, is meant to refer to an agent that mimics or upregulates (e.g. potentiates or supplements) an htrb bioactivity. An htrb agonist can be a wild-type htrb protein or derivative thereof having at least one bioactivity of the wild-type htrb , e.g. an AP-1 activation inhibitory activity. An htrb therapeutic can also be a compound that upregulates expression of an htrb gene or which increases at least one bioactivity of an htrb protein. An agonist can also be a compound which increases the interaction of an htrb polypeptide with another molecule, e.g, an IL-1 type I or type II receptor. Such an agonist may, for example, increase the binding of htrb to a MAPK, thereby promoting an htrb -dependent blocking of a MAPK mediated inflammatory signal. Alternatively, an htrb agonist may increase the binding of htrb to an MAPK.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. Frequently occurring sequence variations include transition mutations (i.e. purine to purine substitutions and pyrimidine to pyrimidine substitutions, e.g. A to G or C to T), transversion mutations (i.e. purine to pyrimidine and pyrimidine to purine substitutions, e.g. A to T or C to G), and alteration in repetitive DNA sequences (e.g. expansions and contractions of trinucleotide repeat and other tandem repeat sequences). An allele of a gene can also be a form of a gene containing a mutation. The term “allelic variant of a polymorphic region of an htrb gene” refers to a region of an htrb gene having one or several nucleotide sequences found in that region of the gene in other individuals.

“Antagonist” as used herein is meant to refer to an agent that downregulates (e.g. suppresses or inhibits) at least one htrb bioactivity. An htrb antagonist can be a compound which inhibits or decreases the interaction between an htrb protein and another molecule, e.g., a MAPK. An antagonist can also be a compound that down-regulates expression of an htrb gene or which reduces the amount of htrb protein present. The htrb antagonist can be a dominant negative form of an htrb polypeptide, e.g., a form of an htrb polypeptide which is capable of interacting with a target peptide. The htrb antagonist can also be a nucleic acid encoding a dominant negative form of an htrb polypeptide, an htrb antisense nucleic acid, or a ribozyme capable of interacting specifically with an htrb RNA. Yet other htrb antagonists are molecules which bind to an htrb polypeptide and inhibit its action. Such molecules include peptides, e.g., forms of htrb target peptides which do not have biological activity, and which inhibit binding to htrb target molecules, such as an AP-1 transcription factor.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

A disease, disorder, or condition “associated with” or “characterized by” an aberrant expression of a nucleic acid refers to a disease, disorder, or condition in a subject which is caused by, contributed to by, or causative of an aberrant level of expression of a nucleic acid.

As used herein the term “bioactive fragment of an htrb polypeptide” refers to a fragment of a full-length htrb polypeptide, wherein the fragment specifically mimics or antagonizes the activity of a wild-type htrb polypeptide. The bioactive fragment preferably is a fragment capable of interacting with a MAPK.

“Biological activity” or “bioactivity” or “activity” or “biological function”, which are used interchangeably, for the purposes herein means an effector or antigenic function that is directly or indirectly performed by an htrb polypeptide (whether in its native or denatured conformation), or by any subsequence thereof. Biological activities include binding to a target peptide, e.g., a MAPK, preferably an ERK. An htrb bioactivity can be modulated by directly affecting an htrb polypeptide. Alternatively, an htrb bioactivity can be modulated by modulating the level of an htrb polypeptide, such as by modulating expression of an htrb gene.

The term “biomarker” refers a biological molecule, e.g., a nucleic acid, peptide, hormone, etc., whose presence or concentration can be detected and correlated with a known condition, such as a disease state.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject htrb polypeptides with a second amino acid sequence defining a domain (e.g. polypeptide portion) foreign to and not substantially homologous with any domain of an htrb polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-htrb-Y, wherein htrb represents a portion of the polypeptide which is derived from an htrb polypeptide, and X and Y are independently absent or represent amino acid sequences which are not related to an htrb sequence in an organism, including naturally occurring mutants.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

As is well known, genes may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding an htrb polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids shown in, for example, SEQ ID Nos. 1 and 3, due to the degeneracy of the genetic code.

The term “haplotype” as used herein is intended to refer to a set of alleles that are inherited together as a group (are in linkage disequilibrium) at statistically significant levels (p_{corr}<0.05). As used herein, the phrase “an htrb haplotype” refers to a haplotype in the htrb loci which may include polymorphic variations of htrb gene sequences.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the htrb sequences of the present invention.

As used herein, the term “htrb ” refers to a mammalian homolog of a Drosophila tribbles gene, or an equivalent thereof. As used herein, the term htrb is used interchangeably with the term “SKIP)” gene or protein, a second name for htrb based upon its stress kinase inhibitory activity (i.e. SKIP from Stress Kinase Inhibitor Protein, wherein htrb-1 is used interchangably with SKIP-1). Further as used herein, the htrb-3 gene is also referred to as the SKIP-3 gene.

The term “htrb nucleic acid” refers to a nucleic acid encoding an htrb protein, such as nucleic acids having SEQ ID Nos. 1 or 3 or fragments thereof, a complement thereof, and derivatives thereof.

The terms “htrb polypeptide” and “htrb protein” are intended to encompass polypeptides comprising the amino acid sequence shown as SEQ ID No. 1 or 3 et al. or fragments thereof, and homologs thereof and include agonist and antagonist polypeptides.

The term “htrb binding partner” or “htrb BP” refers to various cell proteins which bind to an htrb protein.

The term “htrb therapeutic” refers to various forms of htrb polypeptides, as well as peptidomimetics, nucleic acids, or small molecules, which can modulate at least one activity of an htrb polypeptide, e.g., interaction with an htrb receptor interaction with and/or an htrb coreceptor, by mimicking or potentiating (agonizing) or inhibiting (antagonizing) the effects of a naturally-occurring htrb polypeptide. An htrb therapeutic which mimics or potentiates the activity of a wild-type htrb polypeptide is an “htrb agonist”. Conversely, an htrb therapeutic which inhibits the activity of a wild-type htrb polypeptide is an “htrb antagonist”.

“Increased risk” refers to a statistically higher frequency of occurrence of the disease or condition in an individual carrying a particular polymorphic allele in comparison to the frequency of occurrence of the disease or condition in a member of a population that does not carry the particular polymorphic allele.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject htrb polypeptides preferably includes no more than 10 kilobases kb) of nucleic acid sequence which naturally immediately flanks the htrb gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.). In preferred embodimbents, the “knock-out” gene locus corresponding to the modified endogenous gene no longer encodes a functional polypeptide activity and is said to be a “null” allele. Accordingly, knock-out transgenic animals of the present invention include those carrying one htrb gene null mutation, such as htrb null allele heterozygous animals, and those carrying two htrb gene null mutations, such as htrb null allele homozygous animals.

A “knock-out construct” refers to a nucleic acid sequence that can be used to decrease or suppress expression of a protein encoded by endogenous DNA sequences in a cell. In a simple example, the knock-out construct is comprised of a gene, such as the htrb gene, with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: htrb 5′/neo/htrb 3′, where htrb 5′ and htrb 3′, refer to genomic or cDNA sequences which are, respectively, upstream and downstream relative to a portion of the htrb gene and where neo refers to a neomycin resistance gene. In another knock-out construct, a second selectable marker is added in a flanking position so that the gene can be represented as: htrb/neo/htrb/TK, where TK is a thymidine kinase gene which can be added to either the htrb 5′ or the htrb 3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

“Linkage disequilIbrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage equilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are 100% in linkage disequilibrium with the two most frequent alleles of IL-1RN (VNTR), which are allele 1 and allele 2.

The term “marker” refers to a sequence in the genome that is known to vary among individuals. For example, the IL-1RN gene has a marker that consists of a variable number of tandem repeats (VNTR).

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant htrb genes is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x. The nucleotide sequences and complementary sequences thereof are always given in the 5′ to 3′ direction.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence of a sequence shown in one of SEQ ID Nos. of the invention. Nucleic acids at least 90%, more preferably 95%, and most preferably at least about 98-99% identical with a nucleic sequence represented in one of SEQ ID Nos: 1-2 are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987)25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The term “propensity to disease,” also “predisposition” or “susceptibility” to disease or any similar phrase, means that certain htrb locus polymorphic alleles are hereby discovered to be associated with or predictive of a particular disease. The alleles are thus over-represented in frequency in individuals with disease as compared to healthy individuals. Thus, these alleles can be used to predict disease even in pre-symptomatic or pre-diseased individuals.

The terms “protein”, “polypeptide” and “peptide” are used interchangeably herein when referring to a gene product.

The term “recombinant protein” refers to a polypeptide of the present invention which is produced by recombinant DNA techniques, wherein generally, DNA encoding an htrb polypeptide is inserted into a suitable expression vector which is in turn used to transform a host cell to produce the heterologous protein. Moreover, the phrase “derived from”, with respect to a recombinant htrb gene, is meant to include within the meaning of “recombinant protein” those proteins having an amino acid sequence of a native htrb polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate an htrb bioactivity.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably an htrb gene. “Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the htrb genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of htrb polypeptide.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of an htrb polypeptide or, in the case of anti-sense expression from the transferred gene, the expression of a naturally-occurring form of the htrb polypeptide is disrupted.

As used herein, the term “transgene” means a nucleic acid sequence (encoding, e.g., one of the htrb polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the htrb polypeptides, e.g. either agonistic or antagonistic forms. However, transgenic animals in which the recombinant htrb gene is silent are also contemplated, as for example, the FLP or CRE recombinase dependent constructs described below. Moreover, “transgenic animal” also includes those recombinant animals in which gene disruption of one or more htrb genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

4.3. Nucleic Acids of the Present Invention

The invention provides htrb nucleic acids, homologs thereof, and portions thereof. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferbly 95% and even more preferably at least 99% homologous with a nucleotide sequence of an htrb gene, e.g., such as a sequence shown in one of SEQ ID Nos: 1 or 3 or complement thereof of the htrb nucleic acids having the GenBank Accession Nos.: htrb-1 (AF250310) and htrb-3 (AF250311). Nucleic acids at least 90%, more preferably 95%, and most preferably at least about 98-99% identical with a nucleic sequence represented in one of SEQ ID Nos. 1 or 3 or complement thereof are of course also within the scope of the invention. In preferred embodiments, the nucleic acid is mammalian and in particularly preferred embodiments, includes all or a portion of the nucleotide sequence corresponding to the coding region of the nucleic acid set forth in SEQ ID No. 1 or 3 which correspond to the htrb-1 and htrb-3 ORF respectively.

The invention further provides an evolutionarily conserved nucleic acid sequence found in the 3′ UTR (untranslated region) of the htrb transcript encoded by both mammalian homologs of the htrb gene, which is described in further detail in the examples which follow.

The invention also pertains to isolated nucleic acids comprising a nucleotide sequence encoding htrb polypeptides, variants and/or equivalents of such nucleic acids. The term equivalent is understood to include nucleotide sequences encoding functionally equivalent htrb polypeptides or functionally equivalent peptides having an activity of an htrb protein such as described herein. Equivalent nucleotide sequences will include sequences that differ by one or more nucleotide substitution, addition or deletion, such as allelic variants; and will, therefore, include sequences that differ from the nucleotide sequence of the htrb gene shown in SEQ ID Nos. 1, 2, 3, or 4 due to the degeneracy of the genetic code.

Preferred nucleic acids are vertebrate htrb nucleic acids. Particularly preferred vertebrate htrb nucleic acids are mammalian. Regardless of species, particularly preferred htrb nucleic acids encode polypeptides that are at least 60%, 65%, 70%, 72%, 74%, 76%, 78%, 80%, 90%, or 95% similar or identical to an amino acid sequence of a vertebrate htrbprotein. In one embodiment, the nucleic acid is a cDNA encoding a polypeptide having at least one bio-activity of the subject htrb polypeptide. Preferably, the nucleic acid includes all or a portion of the nucleotide sequence corresponding to the nucleic acid of SEQ ID Nos. 1 or 3.

Still other preferred nucleic acids of the present invention encode an htrb polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

Another aspect of the invention provides a nucleic acid which hybridizes under stringent conditions to a nucleic acid represented by SEQ ID Nos. 1 or 3 or complement thereof or the nucleic acids having ATCC Designation No. XXXXXX or No. XXXXXX. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, an htrb nucleic acid of the present invention will bind to one of SEQ ID Nos. 1 or 3 or complement thereof under moderately stringent conditions, for example at about 2.0×SSC and about 40° C. In a particularly preferred embodiment, an htrb nucleic acid of the present invention will bind to one of SEQ ID Nos. 1 or 3 or complement thereof under high stringency conditions. In another particularly preferred embodiment, an htrb nucleic acid sequence of the present invention will bind to a portion of one of SEQ ID Nos. 1 or 3 that corresponds to the htrb ORF nucleic acid sequences, under high stringency conditions.

Nucleic acids having a sequence that differs from the nucleotide sequences shown in one of SEQ ID Nos. 1 or 3 of the invention or complement thereof due to degeneracy in the genetic code are also within the scope of the invention. Such nucleic acids encode functionally equivalent peptides (i.e., peptides having a biological activity of an htrb polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of an htrb polypeptide. However, it is expected that DNA sequence polymorphisms that do lead to changes in the amino acid sequences of the subject htrb polypeptides will exist among mammals. One skilled in the art will appreciate that these variations in one or more nucleotides (e.g., up to about 3-5% of the nucleotides) of the nucleic acids encoding polypeptides having an activity of an htrb polypeptide may exist among individuals of a given species due to natural allelic variation.

4.3.1 Probes and Primers

The nucleotide sequences determined from the cloning of htrb genes from mammalian organisms will further allow for the generation of probes and primers designed for use in identifying and/or cloning other htrb homologs in other cell types, e.g., from other tissues, as well as htrb homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from SEQ ID Nos. 1 or 3 of the invention. For instance, primers based on the nucleic acid represented in SEQ ID Nos. 1 or 3 can be used in PCR reactions to clone htrb polypeptide encoding genes.

In preferred embodiments, the htrb primers are designed so as to optimize specificity and avoid secondary structures which affect the efficiency of priming. Optimized PCR primers of the present invention are designed so that “upstream” and “downstream” primers have approximately equal melting temperatures such as can be estimated using the formulae: Tₘ=81.5 C−16.6(log₁₀ [Na⁺])+0.41(% G+C)−0.63 (% formamide)−(600/length); or Tₘ(C)=2(A/T)+4(G/C). Optimized htrb primers may also be designed by using various programs, such as “Primer3” provided by the Whitehead Institute for Biomedical Research at http://www-genome.wi.mit.edu/cgi-bin/primer/primer3.cgi.

In preferred embodiments, the htrb probes and primers can be used to detect htrb locus polymorphisms which occur within and surrounding the htrb gene sequence. Genetic variations within the htrb locus may be associated with the likelihood of the development of a number of human diseases and conditions, such as inflammatory and autoimmune diseases in which htrb encoded polypeptides play an important etiological role. Accordingly the invention provides probes and primers for htrb locus polymorphisms, including polymorphisms associated with the human and mouse htrb gene. PCR primers of the invention include those which flank an htrb human polymorphism and allow amplification and analysis of this region of the genome. Analysis of polymorphic allele identity may be conducted, for example, by direct sequencing or by the use of allele-specific capture probes or by the use of molecular beacon probes. Alternatively, the polymorphic allele may allow for direct detection by the creation or elimination of a restriction endonuclease recognition site(s) within the PCR product or after an appropriate sequence modification is designed into at least one of the primers such that the altered sequence of the primer, when incorporated into the PCR product resulting from amplification of a specific htrb polymorphic allele, creates a unique restriction site in combination with at least one allele but not with at least one other allele of that polymorphism. htrb polymorphisms corresponding to variable number of tandem repeat (VNTR) polymorphisms may be detected by the electrophoretic mobility and hence size of a PCR product obtained using primers which flank the VNTR. Still other htrb polymorphisms corresponding to restriction fragment length polymorphisms (RFLPs) may be detected directly by the mobility of bands on a Southern blot using appropriate htrb locus probes and genomic DNA or cDNA obtained from an appropriate sample organism such as a human or a non-human animal.

Likewise, probes based on the subject htrb sequences can be used to detect transcripts or genomic sequences encoding the same or homologous proteins, for use, e.g, in prognostic or diagnostic assays (further described below). The invention provides probes which are common to alternatively spliced variants of the htrb transcript, such as those corresponding to at least 12 consecutive nucleotides complementary to a sequence found in any of SEQ ID Nos. 1 or 3 of the invention. In addition, the invention provides probes which hybridize specifically to alternatively spliced forms of the htrb transcript. Probes and primers can be prepared and modified, e.g., as previously described herein for other types of nucleic acids.

4.3.2 Antisense, Ribozyme and Triplex Techniques

Another aspect of the invention relates to the use of the isolated nucleic acid in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject htrb proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes an htrb protein. Alternatively, the antisense construct is an oligonucleotide probe which is generated ex vivo and which, when introduced into the cell causes inhibition of expression by hybridizing with the mRNA and/or genomic sequences of an htrb gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in anti sense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the htrb nucleotide sequence of interest, are preferred.

Antisense approaches involve the design of oligonucleotides (either DNA or RNA) that are complementary to htrb mRNA. The antisense oligonucleotides will bind to the htrb mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of an htrb gene could be used in an antisense approach to inhibit translation of endogenous htrb mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of htrb mRNA, antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their ability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides maybe synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to the htrb coding region sequence can be used, those complementary to the transcribed untranslated region and to the region comprising the initiating methionine are most preferred.

The antisense molecules can be delivered to cells which express htrb in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous htrb transcripts and thereby prevent translation of the htrb mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive and can include but not be limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Ribozyme molecules designed to catalytically cleave htrb mRNA transcripts can also be used to prevent translation of htrb mRNA and expression of htrb (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy htrb mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. For example, there are a number of potential hammerhead ribozyme cleavage sites within the nucleotide sequence of human htrb-1 and htrb-3. Preferably the ribozyme is engineered so that the cleavage recognition site is located near the 5′ end of the htrb mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in an htrb gene.

As in the antisense approach, the ribozymes can be composed of modified oligonucleotides (e.g., for improved stability, targeting, etc.) and should be delivered to cells which express the htrb gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous htrb messages and inhibit translation. Because ribozymes unlike antisense molecules, are catalytic, a lower intracellular concentration is required for efficiency.

Endogenous htrb gene expression can also be reduced by inactivating or “knocking out” the htrb gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional htrb (or a completely unrelated DNA sequence) flanked by DNA homologous to the endogenous htrb gene (either the coding regions or regulatory regions of the htrb gene) can be used, with or without a selectable marker and/or a negative selectable marker, to transfect cells that express htrb in vivo. Insertion of the DNA construct, via targeted homologous recombination, results in inactivation of the htrb gene. Such approaches are particularly suited in the agricultural field where modifications to ES (embryonic stem) cells can be used to generate animal offspring with an inactive htrb (e.g., see Thomas & Capecchi 1987 and Thompson 1989, supra). However this approach can be adapted for use in humans provided the recombinant DNA constructs are directly administered or targeted to the required site in vivo using appropriate viral vectors.

Alternatively, endogenous htrb gene expression can be reduced by targeting deoxyribonucleotide sequences complementary to the regulatory region of the htrb gene (i.e., the htrb promoter and/or enhancers) to form triple helical structures that prevent transcription of the htrb gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

4.3.3. Vectors Encoding htrb Proteins and htrb Expressing Cells

The invention further provides plasmids and vectors encoding an htrb protein, which can be used to express an htrb protein in a host cell. The host cell may be any prokaryotic or eukaryotic cell. Thus, a nucleotide sequence derived from the cloning of mammalian htrb proteins, encoding all or a selected portion of the full-length protein, can be used to produce a recombinant form of an htrb polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures well known in the art.

Vectors that allow expression of a nucleic acid in a cell are referred to as expression vectors. Typically, expression vectors used for expressing an htrb protein contain a nucleic acid encoding an htrb polypeptide, operably linked to at least one transcriptional regulatory sequence. Regulatory sequences are art-recognized and are selected to direct expression of the subject htrb proteins. Transcriptional regulatory sequences are described in Goeddel; Gene Expression Technology: Methods in Enzymology 185, Academic Press, San Diego, Calif. (1990). In one embodiment, the expression vector includes a recombinant gene encoding a peptide having an agonistic activity of a subject htrb polypeptide, or alternatively, encoding a peptide which is an antagonistic form of an htrb protein.

Suitable vectors for the expression of an htrb polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used. In an illustrative embodiment, an htrb polypeptide is produced recombinantly utilizing an expression vector generated by sub-cloning the coding sequence of one of the htrb genes represented in SEQ ID Nos. 1 or 3.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In some instances, it may be desirable to express the recombinant htrb polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III)

When it is desirable to express only a portion of an htrb protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing htrb derived polypeptides in a host which produces MAP (e.g., E. coli or CM89 or S. cerevisiae), or in vitro by use of purified MAP (e.g., procedure of Miller et al., supra).

Moreover, the gene constructs of the present invention can also be used as part of a gene therapy protocol to deliver nucleic acids encoding either an agonistic or antagonistic form of one of the subject htrb proteins. Thus, another aspect of the invention features expression vectors for in vivo or in vitro transfection and expression of an htrb polypeptide in particular cell types so as to reconstitute the function of, or alternatively, abrogate the function of htrb in a tissue. This could be desirable, for example, when the naturally-occurring form of the protein is misexpressed or the natural protein is mutated and less active.

In addition to viral transfer methods, non-viral methods can also be employed to cause expression of a subject htrb polypeptide in the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject htrb polypeptide gene by the targeted cell. Exemplary targeting means of this type include liposomal derived systems, poly-lysine conjugates, and artificial viral envelopes.

In other embodiments transgenic animals, described in more detail below could be used to produce recombinant proteins.

4.4. Polypeptides of the Present Invention

The present invention makes available isolated htrb polypeptides which are isolated from, or otherwise substantially free of other cellular proteins. The term “substantially free of other cellular proteins” (also referred to herein as “contaminating proteins”) or “substantially pure or purified preparations” are defined as encompassing preparations of htrb polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Preferred htrb proteins of the invention have an amino acid sequence which is at least about 60%, 65%, 66%, 67%, 68%, 69%, 70%, 71%, 72%, 73%, 74%, 75%, 76%, 77%, 78%, 79%, 80%, 85%, 90%, or 95% identical or homologous to an amino acid sequence of SEQ ID No. 2 or 4. Even more preferred htrb proteins comprise an amino acid sequence of at least 10, 20, 30, or 50 residues which is at least about 70, 80, 90, 95, 97, 98, or 99% homologous or identical to an amino acid sequence of SEQ ID Nos. 2 or 4. Such proteins can be recombinant proteins, and can be, e.g., produced in vitro from nucleic acids comprising a nucleotide sequence set forth in SEQ ID Nos. 1 or 3, or another nucleic acid of the invention or homologs thereof. For example, recombinant polypeptides preferred by the present invention can be encoded by a nucleic acid, which is at least 85% homologous and more preferably 90% homologous and most preferably 95% homologous with a nucleotide sequence set forth in a SEQ ID Nos. 1 or 3 of the invention. Polypeptides which are encoded by a nucleic acid that is at least about 98-99% homologous with the sequence of SEQ ID No. 1 or 3 of the invention are also within the scope of the invention.

In a preferred embodiment, an htrb protein of the present invention is a mammalian htrb protein. In a particularly preferred embodiment an htrb protein is set forth as SEQ ID No. 2 or SEQ ID No. 4. In particularly preferred embodiments, an htrb protein has an htrb bioactivity. It will be understood that certain post-translational modifications, e.g., phosphorylation and the like, can increase the apparent molecular weight of the htrb protein relative to the unmodified polypeptide chain.

The invention also features protein isoforms encoded by splice variants of the present invention. Such isoforms may have biological activities identical to or different from those possessed by the htrb proteins specified by Nos. 2 or 4. Such isoforms may arise, for example, by alternative splicing of one or more htrb gene transcripts.

htrb polypeptides preferably are capable of functioning as either an agonist or antagonist of at least one biological activity of a wild-type (“authentic”) htrb protein of the appended sequence listing. The term “evolutionarily related to”, with respect to amino acid sequences of htrb proteins, refers to both polypeptides having amino acid sequences which have arisen naturally, and also to mutational variants of human htrb polypeptides which are derived, for example, by combinatorial mutagenesis.

Full length proteins or fragments corresponding to one or more particular motifs and/or domains or to arbitrary sizes, for example, at least 5, 10, 20, 25, 50, 75 and 100, amino acids in length are within the scope of the present invention.

For example, isolated htrb polypeptides can be encoded by all or a portion of a nucleic acid sequence shown in any of SEQ ID Nos. 1 or 3. Isolated peptidyl portions of htrb proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an htrb polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) htrb protein.

An htrb polypeptide can be a membrane bound form or a soluble form. A preferred soluble htrb polypeptide is a polypeptide which does not contain a hydrophobic signal sequence domain. Such proteins can be created by genetic engineering by methods known in the art. The solubility of a recombinant polypeptide may be increased by deletion of hydrophobic domains, such as predicted transmembrane domains, of the wild type protein.

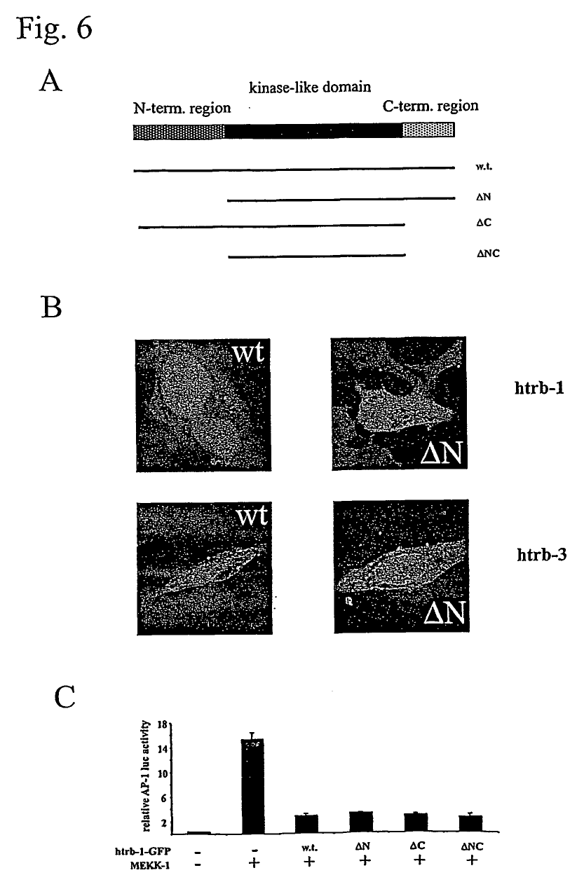

In general, polypeptides referred to herein as having an activity (e.g., are “bioactive”) of an htrb protein are defined as polypeptides which include an amino acid sequence encoded by all or a portion of the nucleic acid sequences shown in one of SEQ ID No. 1 or 3 and which mimic or antagonize all or a portion of the biological/biochemical activities of a naturally occurring htrb protein. Examples of such biological activity include a region of conserved structure referred to as the htrb conserved domain (see FIG. 6A, htrb NC construct).

Other biological activities of the subject htrb proteins will be reasonably apparent to those skilled in the art. According to the present invention, a polypeptide has biological activity if it is a specific agonist or antagonist of a naturally-occurring form of an htrb protein.

Assays for determining whether a compound, e.g, a protein, such as an htrb protein or variant thereof, has one or more of the above biological activities include those assays, well known in the art, which are used for assessing htrb agonist and htrb antagonist activities. For example, the ability of recombinant htrb polypeptide to block activation of an AP-1 reporter construct. In contrast, the ability of recombinant htrb polypeptides to interfere with cytokine induced activation of interleukin-8 gene expression is indicative of htrb antagonist activity.

Other preferred proteins of the invention are those encoded by the nucleic acids set forth in the section pertaining to nucleic acids of the invention. In particular, the invention provides fusion proteins, e.g., htrb immunoglobulin fusion proteins. Such fusion proteins can provide, e.g., enhanced stability and solubility of htrb proteins and may thus be useful in therapy. Fusion proteins can also be used to produce an immunogenic fragment of an htrb protein. For example, the VP6 capsid protein of rotavirus can be used as an immunologic carrier protein for portions of the htrb polypeptide, either in the monomeric form or in the form of a viral particle. The nucleic acid sequences corresponding to the portion of a subject htrb protein to which antibodies are to be raised can be incorporated into a fusion gene construct which includes coding sequences for a late vaccinia virus structural protein to produce a set of recombinant viruses expressing fusion proteins comprising htrb epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of an htrb protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of an htrb polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of htrb proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the htrb polypeptides of the present invention. For example, htrb polypeptides can be generated as glutathione-S-transferase (GST-fusion) proteins. Such GST-fusion proteins can enable easy purification of the htrb polypeptide, as for example by the use of glutathione-derivatized matrices (see, for example, Current Protocols in Molecular Biology, eds. Ausubel et al. (N.Y.: John Wiley & Sons, 1991)). Additionally, fusion of htrb polypeptides to small epitope tags, such as the FLAG or hemagluttinin tag sequences, can be used to simplify immunological purification of the resulting recombinant polypeptide or to facilitate immunological detection in a cell or tissue sample. Fusion to the green fluorescent protein, and recombinant versions thereof which are known in the art and available commercially, may further be used to localize htrb polypeptides within living cells and tissue.

The present invention further pertains to methods of producing the subject htrb polypeptides. For example, a host cell transfected with a nucleic acid vector directing expression of a nucleotide sequence encoding the subject polypeptides can be cultured under appropriate conditions to allow. expression of the peptide to occur. Suitable media for cell culture are well known in the art. The recombinant htrb polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant htrb polypeptide is a fusion protein containing a domain which facilitates its purification, such as GST fusion protein.

Moreover, it will be generally appreciated that, under certain circumstances, it may be advantageous to provide homologs of one of the subject htrb polypeptides which function in a limited capacity as one of either an htrb agonist (mimetic) or an htrb antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of htrb proteins.

Homologs of each of the subject htrb proteins can be generated by mutagenesis, such as by discrete point mutation(s), or by truncation. For instance, mutation can give rise to homologs which retain substantially the same, or merely a subset, of the biological activity of the htrb polypeptide from which it was derived. Alternatively, antagonistic forms of the protein can be generated which are able to inhibit the flnction of the naturally occurring form of the protein, such as by competitively binding to an htrb receptor.

The recombinant htrb polypeptides of the present invention also include homologs of the wildtype htrb proteins, such as versions of those protein which are resistant to proteolytic cleavage, as for example, due to mutations which alter ubiquitination or other enzymatic targeting associated with the protein.

htrb polypeptides may also be chemically modified to create htrb derivatives by forming covalent or aggregate conjugates with other chemical moieties, such as glycosyl groups, lipids, phosphate, acetyl groups and the like. Covalent derivatives of htrb proteins can be prepared by linking the chemical moieties to functional groups on amino acid sidechains of the protein or at the N-terminus or at the C-terminus of the polypeptide.

Modification of the structure of the subject htrb polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the htrb polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, WH Freeman and Co.: 1981). Whether a change in the amino acid sequence of a peptide results in a functional htrb homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject htrb proteins as well as truncation mutants, and is especially useful for identifying potential variant sequences (e.g., homologs). The purpose of screening such combinatorial libraries is to generate, for example, novel htrb homologs which can act as either agonists or antagonist, or alternatively, possess novel activities all together. Thus, combinatorially-derived homologs can be generated to have an increased potency relative to a naturally occurring form of the protein.

In one embodiment, the variegated htrb libary of htrb variants is generated by combinatorial mutagenesis at the nucleic acid level, and is encoded by a variegated gene htrb library. For instance, a mixture of synthetic oligonucleotides can be enzymatically ligated into gene sequences such that the degenerate set of potential htrb sequences are expressible as individual polypeptides, or alternatively, as a set of larger fusion proteins (e.g., for phage display) containing the set of htrb sequences therein.

There are many ways by which such libraries of potential htrb homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential htrb sequences. The synthesis of degenerate oligonucleotides is well known in the art (see for example, Narang, SA (1983) Tetrahedron 39:3; Itakura et al. (1981) Recombinant DNA, Proc 3^{3 rd} Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for an htrb clone in order to generate a variegated population of htrb fragments for screening and subsequent selection of bioactive fragments. A variety of techniques are known in the art for generating such 1, including chemical synthesis. In one embodiment, a library of coding sequence fragments can be generated by (i) treating a double stranded PCR fragment of an htrb coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of htrb homologs. The most widely used techniques for screening large gene libraries typically comprises cloning the gene library into replicable expression vectors, transforming appropriate cells with the resulting libraries of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate htrb sequences created by combinatorial mutagenesis techniques. Combinatorial mutagenesis has a potential to generate very large libraries of mutant proteins, e.g., in the order of 1026 molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the htrb proteins to generate mimetics, e.g., peptide or non-peptide agents, such as small molecules, which are able to disrupt binding of an htrb polypeptide of the present invention with a molecule, e.g. target peptide. Thus, such mutagenic techniques as described above are also usefull to map the determinants of the htrb proteins which participate in protein-protein interactions involved in, for example, binding of the subject htrb polypeptide to a target peptide. To illustrate, the critical residues of a subject htrb polypeptide which are involved in molecular recognition of its receptor can be determined and used to generate htrb derived peptidomimetics or small molecules which competitively inhibit binding of the authentic htrb protein with that moiety. By employing, for example, scanning mutagenesis to map the amino acid residues of the subject htrb proteins which are involved in binding other proteins, peptidomimetic compounds can be generated which mimic those residues of the htrb protein which facilitate the interaction. Such mimetics may then be used to interfere with the normal function of an htrb protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffinan et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9^{th} American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun126:419; and Dann et al. (1986) Biochem Biophys Res Commun 134:71).

4.5. Anti-htrb Antibodies and Uses Therefor

Another aspect of the invention pertains to an antibody specifically reactive with a mammalian htrb protein, e.g., a wild-type or mutated htrb protein. For example, by using immunogens derived from an htrb protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a mammalian htrb polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of an htrb protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determiinants of an htrb protein of a mammal, e.g., antigenic determinants of a protein set forth in SEQ ID No. 2 or 4 or closely related homologs (e.g., at least 90% homologous, and more preferably at least 94% homologous).

Following immunization of an animal with an antigenic preparation of an htrb polypeptide, anti-htrb antisera can be obtained and, if desired, polyclonal anti-htrb antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique originally developed by Kohler and Milstein ((1975) Nature, 256: 495-497), the human B cell hybridoma technique (Kozbar et al., (1983) Inmunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian htrb polypeptide of the present invention and monoclonal antibodies isolated from a culture comprising such hybridoma cells. In one embodiment anti-human htrb antibodies specifically react with the protein encoded by a nucleic acid having SEQ ID No. 1 or 3.

The term antibody as used herein is intended to include fragments thereof which are also specifically reactive with one of the subject mammalian htrb polypeptides. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific, single-chain, and chimeric and humanized molecules having affinity for an htrb protein conferred by at least one CDR region of the antibody. In preferred embodiments, the antibody further comprises a label attached thereto and able to be detected, (e.g., the label can be a radioisotope, fluorescent compound, enzyme or enzyme co-factor).

Anti-htrb antibodies can be used, e.g., to monitor htrb protein levels in an individual for determining, e.g., whether a subject has a disease or condition associated with an aberrant htrb protein level, or allowing determination of the efficacy of a given treatment regimen for an individual afflicted with such a disorder. The level of htrb polypeptides may be measured from cells in bodily fluid, such as in blood samples.

Another application of anti-htrb antibodies of the present invention is in the immunological screening of cDNA libraries constructed in expression vectors such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of an htrb protein, e.g., other orthologs of a particular htrb protein or other paralogs from the same species, can-then be detected with antibodies, as, for example, reacting nitrocellulose filters lifted from infected plates with anti-htrb antibodies. Positive phage detected by this assay can then be isolated from the infected plate. Thus, the presence of htrb homologs can be detected and cloned from other animals, as can alternate isoforms (including splice variants) from humans.

4.6. Transgenic Animals

The invention further provides for transgenic animals, which can be used for a variety of purposes, e.g., to identify htrb therapeutics. Transgenic animals of the invention include non-human animals containing a heterologous htrb gene or fragment thereof under the control of an htrb promoter or under the control of a heterologous promoter. Accordingly, the transgenic animals of the invention can be animals expressing a transgene encoding a wild-type htrb protein or fragment thereof or variants thereof, including mutants and polymorphic variants thereof. Such animals can be used, e.g., to determine the effect of a difference in amino acid sequence of an htrb protein from the sequence set forth in SEQ ID Nos. 2 or 4, such as a polymorphic difference. These animals can also be used to determine the effect of expression of an htrb protein in a specific site or for identifying htrb therapeutics or confirming their activity in vivo.

In one aspect, the invention provides transgenic non-human organisms and cell lines for use in the in vivo screening and evaluation of drugs or other therapeutic regimens useful in the treatment of inflammatory disorders. In one embodiment, the invention is a transgenic animal with a targeted disruption in an interleukin-1 gene. In particular, the gene is the htrb gene. The animal may be chimeric, heterozygotic or homozygotic for the disrupted gene. Homozygotic knock-out htrb mammals provide a model for studying inflammatory conditions, such as rheumatoid arthritis, inflammatory bowel disorder, Type I diabetes, psoriasis, osteoporosis, nephropathy in diabetes mellitus, alopecia areata, Graves disease, systemic lupus erythematosus, lichen sclerosis, ulcerative colitis, coronary artery disease, arteritic disorders, diabetic retinopathy, low birth weight, pregnancy complications, severe periodontal disease, psoriasis and insulin dependent diabetes, but is particularly characterized by arteritic lesions. The targeted disruption may be anywhere in the gene, subject only to the requirement that it inhibit production of functional htrb protein. In a preferred embodiment, the disruption removes the entire htrb coding sequence such as that of htrb-1 contained in SEQ ID No. 1. The transgenic animal maybe of any species (except human), but is preferably a mammal. In a preferred embodiment, the non-human animal comprising a targeted disruption in the htrb gene, wherein said targeted disruption inhibits production of wild-type htrb polypeptide so that the phenotype of a non-human mammal homozygous for the targeted disruption is characterized by an altered inflammatory response.

In another aspect, the invention features a cell or cell line, which contains a targeted disruption in the htrb gene. In a preferred embodiment, the cell or cell line is an undifferentiated cell, for example, a stem cell, embryonic stem cell, oocyte or embryonic cell.

Yet in a further aspect, the invention features a method of producing a non-human mammal with a targeted disruption in an htrb gene. For example, an htrb knock-out construct can be created with a portion of the htrb gene having an internal portion of said htrb gene replaced by a marker. The knock-out construct can then be transfected into a population of embryonic stem m(ES) cells. Transfected cells can then be selected as expressing the marker. The transfected ES cells can then be introduced into an embryo of an ancestor of said mammal. The embryo can be allowed to develop to term to produce a chimeric mammal with the knock-out construct in its germline. Breeding said chimeric mammal will produce a heterozygous mammal with a targeted disruption in the htrb gene. Homozygotes can be generated by crossing heterozygotes.

In another aspect, the invention features htrb knock-out constructs, which can be used to generate the animals described above. In one embodiment, the htrb construct can comprise a portion of the htrb gene, wherein an internal portion of said htrb gene is replaced by a selectable marker. Preferably, the marker is the neo gene and the portion of the htrb gene is at least 2.5 kb long or 7.0 or 9.5 kb long (including the replaced portion and any htrb flanking sequences). The internal portion preferably covers at least a portion of an exon and in some embodiments it covers all of the exons which encode an htrb polypeptide.

In still another aspect, the invention features methods for testing agents for effectiveness in treating and/or preventing an inflammatory condition. In one embodiment, the method can employ the transgenic animal or cell lines, as described above. For example, a test agent can be administered to the transgenic animal and the ability of the agent to ameliorate the inflammatory condition can be scored as having effectiveness against said inflammatory condition. Any inflammatory condition with an htrb component can be tested using these mammals, but in particular, conditions characterized by arteritic lesions are studied. The method may also be used to test agents that are effective against inflammatory proteins and their downstream components.

The transgenic animals can also be animals containing a transgene, such as reporter gene, under the control of an htrb promoter or fragment thereof. These animals are useful, e.g., for identifying htrb drugs that modulate production of htrb, such as by modulating htrb gene expression. An htrb gene promoter can be isolated, e.g., by screening of a genomic library with an htrb cDNA fragment and characterized according to methods known in the art. In a preferred embodiment of the present invention, the transgenic animal containing said htrb reporter gene is used to screen a class of bioactive molecules known as steroid hormones for their ability to modulate htrb expression. In a more preferred embodiment of the invention, the steroid hormones screened for htrb expression modulating activity belong to the group known as androgens. In a still more preferred embodiment of the invention, the steroid hormone is testosterone or a testosterone analog. Yet other non-human animals within the scope of the invention include those in which the expression of the endogenous htrb gene has been mutated or “knocked out”. A “knock out” animal is one carrying a homozygous or heterozygous deletion of a particular gene or genes. These animals could be useful to determine whether the absence of htrb will result in a specific phenotype, in particular whether these mice have or are likely to develop a specific disease, such as high susceptibility to heart disease or cancer. Furthermore these animals are useful in screens for drugs which alleviate or attenuate the disease condition resulting from the mutation of the htrb gene as outlined below. These animals are also useful for determining the effect of a specific amino acid difference, or allelic variation, in an htrb gene. That is, the htrb knock out animals can be crossed with transgenic animals expressing, e.g., a mutated form or allelic variant of htrb, thus resulting in an animal which expresses only the mutated protein and not the wild-type htrb protein.

In a preferred embodiment of this aspect of the invention, a transgenic htrb knock-out mouse, carrying the mutated htrb locus on one or both of its chromosomes, is used as a model system for transgenic or drug treatment of the condition resulting from loss of htrb expression.

Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a htrb gene of interest in ES cells, these changes can be introduced into the gernlines of animals to generate chimeras. The gene targeting procedure is accomplished by introducing into tissue culture cells a DNA targeting construct that includes a segment homologous to a target htrb locus, and which also includes an intended sequence modification to the htrb genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a htrb gene function through the use of a targeting transgene construct designed to undergo homologous recombination with one or more htrb genomic sequences. The targeting construct can be arranged so that, upon recombination with an element of a htrb gene, a positive selection marker is inserted into (or replaces) coding sequences of the gene. The inserted sequence functionally disrupts the htrb gene, while also providing a positive selection trait. Exemplary htrb targeting constructs are described in more detail below.

Generally, the embryonic stem cells (ES cells ) used to produce the knockout animals will be of the same species as the knockout aninal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]).

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for htrb or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targetted gene, resulting in a transgenic animal which expresses a polypeptide of the targetted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., b-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the abovementioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyaridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the htrb gene knocked out in various tissues of the offspring by probing the Western blot with an antibody against the particular htrb protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a htrb-gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s) .

A htrb transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a htrb protein can be essential for many forms of lineage analysis and can additionally provide a means to assess the effects of, for example, lack of htrb expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject htrb proteins. For example, excision of a target sequence which interferes with the expression of a recombinant htrb gene, such as one which encodes an antagonistic homolog or an antisense transcript, can be designed to activate expression of that gene. This interference with expression of the protein can result from a variety of mechanisms, such as spatial separation of the htrb gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxp sequences. loxp sequences are 34 base pair nucleotide repeat sequences to which the Cre recombinase binds and are required for Cre recombinase mediated genetic recombination. The orientation of loxp sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxp sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant htrb protein can be regulated via control of recombinase expression.

Use of the cre/loxP recombinase system to regulate expression of a recombinant htrb protein requires the construction of a transgenic animal containing transgenes encoding both the Cre recombinase and the subject protein. Animals containing both the Cre recombinase and a recombinant htrb gene can be provided through the construction of “double” transgenic animals. A convenient method for providing such animals is to mate two transgenic animals each containing a transgene, e.g., a htrb gene and recombinase gene.

One advantage derived from initially constructing transgenic animals containing a htrb transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic htrb transgene is silent will allow the study of progeny from that founder in which disruption of htrb mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the htrb transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, an htrb transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed) . In one embodiment, the transgene construct is introduced into a single stage embryo. The zygote is the best target for micro-injection. In the mouse, the male pronucleus reaches the size of approximately 20 micrometers in diameter which allows reproducible injection of 1-2pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a htrb protein (either agonistic or antagonistic), and antisense transcript, or a htrb mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

4.7. Screening Assays for htrb Therapeutics

The invention further provides screening methods for identifying htrb therapeutics, e.g., for treating and/or preventing the development of diseases or conditions caused by, or contributed to by an abnormal htrb activity or which can benefit from a modulation of an htrb activity or protein level. Examples of such diseases, conditions or disorders such as those involving the inflammatory response inluding without limitation: rheumatoid arthritis, inflammatory bowel disorder, Type I diabetes, psoriasis, osteoporosis, nephropathy in diabetes mellitus, alopecia areata, Graves disease, systemic lupus erythematosus, lichen sclerosis, ulcerative colitis, coronary artery disease, arteritic disorders, diabetic retinopathy, low birth weight, pregnancy complications, severe periodontal disease, psoriasis and insulin dependent diabetes, but is particularly characterized by arteritic lesions, cancer e.g., cancers involving the growth of steroid hormone-responsive tumors (e.g. breast, prostate, or testicular cancer), vascular diseases or disorders (e.g. thrombotic stroke, ischemic stroke, as well as peripheral vascular disease resulting from atherosclerotic and thrombotic processes), cardiac disorders (e.g., myocardial infarction, congestive heart failure, unstable angina and ishemic heart disease); and cardiovascular system diseases and disorders (e.g. those resulting from hypertension, hypotension, cardiomyocyte hypertrophy and congestive heart failure) or other diseases conditions or disorders which result from aberrations or alterations of htrb-dependent processes.

An htrb therapeutic can be any type of compound, including a protein, a peptide, peptidomimetic, small molecule, and nucleic acid. A nucleic acid can be, e.g., a gene, an antisense nucleic acid, a ribozyme, or a triplex molecule. An htrb.therapeutic of the invention can be an agonist or an antagonist. Preferred htrb agonists include htrb proteins or derivatives thereof which mimic at least one htrb activity, e.g., fibroblast growth fa,ctor receptor binding or heparin sulfate binding. Other preferred agonists include compounds which are capable of increasing the production of an htrb protein in a cell, e.g., compounds capable of up-regulating the expression of an htrb gene, and compounds which are capable of enhancing an htrb activity and/or the interaction of an htrb protein with another molecule, such as a target peptide. Preferred htrb antagonists include htrb proteins which are dominant negative proteins, which, e.g., are capable of binding to fibroblast growth factor receptors, but not heparin sulfate. Other preferred antagonists include compounds which decrease or inhibit the production of an htrb protein in a cell and compounds which are capable of downregulating expression of an htrb gene, and compounds which are capable of downregulating an htrb activity and/or interaction of an htrb protein with another molecule. In another preferred embodiment, an htrb antagonist is a modified form of a target peptide, which is capable of interacting with the FGFR binding domain of an htrb protein, but which does not have biological activity, e.g., which is not itself a cell surface receptor.

The invention also provides screening methods for identifying htrb therapeutics which are capable of binding to an htrb protein, e.g., a wild-type htrb protein or a mutated form of an htrb protein, and thereby modulate the growth factor activity of htrb or otherwise cause the degradation of htrb. For example, such an htrb therapeutic can be an antibody or derivative thereof which interacts specifically with an htrb protein (either wild-type or mutated).

Thus, the invention provides screening methods for identifying htrb agonist and antagonist compounds, comprising selecting compounds which are capable of interacting with an htrb protein or with a molecule capable of interacting with an htrb protein such as an FGF receptor and/or heparin sulfate and/or a compound which is capable of modulating the interaction of an htrb protein with another molecule, such as a receptor and/or heparin sulfate. In general, a molecule which is capable of interacting with an htrb protein is referred to herein as “htrb binding partner”.

The compounds of the invention can be identified using various assays depending on the type of compound and activity of the compound that is desired. In addition, as described herein, the test compounds can be further tested in animal models. Set forth below are at least some assays that can be used for identifying htrb therapeutics. It is within the skill of the art to design additional assays for identifying htrb therapeutics.

4.7.1. Cell-Free Assays

Cell-free assays can be used to identify compounds which are capable of interacting with an htrb protein or binding partner, to thereby modify the activity of the htrb protein or binding partner. Such a compound can, e.g., modify the structure of an htrb protein or binding partner and thereby effect its activity. Cell-free assays can also be used to identify compounds which modulate the interaction between an htrb protein and an htrb binding partner, such as a target peptide. In a preferred embodiment, cell-free assays for identifying such compounds consist essentially in a reaction mixture containing an htrb protein and a test compound or a library of test compounds in the presence or absence of a binding partner. A test compound can be, e.g., a derivative of an htrb binding partner, e.g., a biologically inactive target peptide, or a small molecule.

Accordingly, one exemplary screening assay of the present invention includes the steps of contacting an htrb protein or functional fragment thereof or an htrb binding partner with a test compound or library of test compounds and detecting the formation of complexes. For detection purposes, the molecule can be labeled with a specific marker and the test compound or library of test compounds labeled with a different marker. Interaction of a test compound with an htrb protein or fragment thereof or htrb binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment; a library of test compounds can be immobilized on a sensor surface, e.g., which forms one wall of a micro-flow cell. A solution containing the htrb protein, functional fragment thereof, htrb analog or htrb binding partner is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) an htrb polypeptide, (ii) an htrb binding partner, and (iii) a test compound; and (b) detecting interaction of the htrb and the htrb binding protein. The htrb polypeptide and htrb binding partner can be produced recombinantly, purified from a source, e.g., plasma, or chemically synthesized, as described herein. A statistically significant change (otentiation or inhibition) in the interaction of the htrb and htrb binding protein in the presence of the test compound, relative to the interaction in the absence of the test compound, indicates a potential agonist (mimetic or potentiator) or antagonist (inhibitor) of htrb bioactivity for the test compound. The compounds of this assay can be contacted simultaneously. Alternatively, an htrb protein can first be contacted with a test compound for an appropriate amount of time, following which the htrb binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified htrb polypeptide or binding partner is added to a composition containing the htrb binding partner or htrb polypeptide, and the formation of a complex is quantitated in the absence of the test compound.

Complex formation between an htrb protein and an htrb binding partner may be detected by a variety of techniques. Modulation of the formation of complexes can be quantitated using, for example, detectably labeled proteins such as radiolabeled, fluorescently labeled, or enzymatically labeled htrb proteins or htrb binding partners, by immunoassay, or by chromatographic detection.

Typically, it will be desirable to immobilize either htrb or its binding partner to facilitate separation of complexes from uncomplexed forms of one or both of the proteins, as well as to accommodate automation of the assay. Binding of htrb to an htrb binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/htrb (GST/htrb) fusion proteins can be adsorbed onto glutathione sepharose beads (Sigma Chemical, St. Louis, Mo.) or glutathione derivatized microtitre plates, which are then combined with the htrb binding partner, e.g. an 35S-labeled htrb binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of htrb protein or htrb binding partner found in the bead fraction quantitated from the gel using standard electrophoretic techniques such as described in the appended examples.

Other techniques for immobilizing proteins on matrices are also available for use in the subject assay. For instance, either htrb or its cognate binding partner can be immobilized utilizing conjugation of biotin and streptavidin. For instance, biotinylated htrb molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with htrb can be derivatized to the wells of the plate, and htrb trapped in the wells by antibody conjugation. As above, preparations of an htrb binding protein and a test compound are incubated in the htrb presenting wells of the plate, and the amount of complex trapped in the well can be quantitated. Exemplary methods for detecting such complexes, in addition to those described above for the GST-immobilized complexes, include immunodetection of complexes using antibodies reactive with the htrb binding partner, or which are reactive with htrb protein and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the htrb binding partner. To illustrate, the htrb binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-htrb antibodies, can be used. Alternatively, the protein to be detected in the complex can be “epitope tagged” in the form of a fusion protein which includes, in addition to the htrb sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

Cell-free assays can also be used to identify compounds which interact with an htrb protein and modulate an activity of an htrb protein. Accordingly, in one embodiment, an htrb protein is contacted with a test compound and the catalytic activity of htrb is monitored. In one embodiment, the abililty of htrb to bind a target molecule is determined. The binding affinity of htrb to a target molecule can be determined according to methods known in the art.

4.7.2. Cell Based Assays

In addition to cell-free assays, such as described above, htrb proteins as provided by the present invention, facilitate the generation of cell-based assays, e.g., for identifying small molecule agonists or antagonists. In one embodiment, a cell expressing an htrb receptor protein on the outer surface of its cellular membrane is incubated in the presence of a test compound alone or in the presence of a test compound and an htrb protein and the interaction between the test compound and the htrb receptor protein or between the htrb protein (preferably a tagged htrb protein) and the htrb receptor is detected, e.g., by using a microphysiometer (McConnell et al. (1992) Science 257:1906). An interaction between the htrb receptor protein and either the test compound or the htrb protein is detected by the microphysiometer as a change in the acidification of the medium. This assay system thus provides a means of identifying molecular antagonists which, for example, function by interfering with htrb—htrb receptor interactions, as well as molecular agonist which, for example, function by activating an htrb receptor.

Cell based assays can also be used to identify compounds which modulate expression of an htrb gene, modulate translation of an htrb mRNA, or which modulate the stability of an htrb mRNA or protein. Accordingly, in one embodiment, a cell which is capable of producing htrb, e.g., a choriocarcinoma cell line such as JEG-3, is incubated with a test compound and the amount of htrb produced in the cell medium is measured and compared to that produced from a cell which has not been contacted with the test compound. The specificity of the compound vis a vis htrb can be confirmed by various control analysis, e.g., measuring the expression of one or more control genes. Compounds which can be tested include small molecules, proteins, and nucleic acids. In particular, this assay can be used to determine the efficacy of htrb antisense molecules or ribozymes.

In another embodiment, the effect of a test compound on transcription of an htrb gene is determined by transfection experiments using a reporter gene operatively linked to at least a portion of the promoter of an htrb gene. A promoter region of a gene can be isolated, e.g., from a genomic library according to methods known in the art. The reporter gene can be any gene encoding a protein which is readily quantifiable, e.g, the luciferase or CAT gene. Such reporter gene are well known in the art.

In preferred embodiments, the invention provides cell-based assays employing the choriocarcinoma cell line JEG-3. Analysis of this cell line has shown that it produces a 17 kDa htrb polypeptide which is immunoprecipitated with rabbit anti-htrb polyclonal antiserum. Accordingly, this cell line can be adapted for screening assays for agents which up-regulate or down-regulate the expression of the htrb gene or otherwise affect the steady-state level of an htrb polypeptide(s) or the efficiency of an htrb polypeptide post-translational activity such as an htrb proteolytic processing event, htrb glycosylation, htrb phosphorylation or htrb secretion.

Other cell-based screening assays which can be employed in the method of the present invention are known or would be apparent to one of skill in the art. For example, htrb agonists and antagonists may be identified by their ability to affect downstream AP-1 dependent transcriptional activation. AP-1 dependent activation occurs by multiple mechanisms.

This invention further pertains to novel agents identified by the above-described screening assays and uses thereof for treatments as described herein.

4.8. Predictive Medicine

The invention further features predictive medicines, which are based, at least in part, on the identity of the novel htrb genes and alterations in the genes and related pathway genes, which affect the expression level and/or function of the encoded htrb protein in a subject.

For example, information obtained using the diagnostic assays described herein (alone or in conjunction with information on another genetic defect, which contributes to the same disease) is useful for diagnosing or confirming that a symptomatic subject (e.g. a subject symptomatic for inflammatory rheumatoid arthritis), has a genetic defect (e.g. in an htrb gene or in a gene that regulates the expression of an htrb gene), which causes or contributes to the particular disease or disorder. Alternatively, the information (alone or in conjunction with information on another genetic defect, which contributes to the same disease) can be used prognostically for predicting whether a non-symptomatic subject is likely to develop a disease or condition, which is caused by or contributed to by an abnormal htrb activity or protein level in a subject. Based on the prognostic information, a doctor can recommend a regimen (e.g. diet or exercise) or therapeutic protocol, useful for preventing or prolonging onset of the particular disease or condition in the individual.

In addition, knowledge of the particular alteration or alterations, resulting in defective or deficient htrb genes or proteins in an individual (the htrb genetic profile), alone or in conjunction with information on other genetic defects contributing to the same disease (the genetic profile of the particular disease) allows customization of therapy for a particular disease to the individual's genetic profile, the goal of “pharmacogenomics”. For example, an individual's htrb genetic profile or the genetic profile of a disease or condition, to which htrb genetic alterations cause or contribute, can enable a doctor to 1) more effectively prescribe a drug that will address the molecular basis of the disease or condition; and 2) better determine the appropriate dosage of a particular drug. For example, the expression level of htrb proteins, alone or in conjunction with the expression level of other genes, known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the htrb or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of htrb as a marker is useful for optimizing effective dose).

These and other methods are described in further detail in the following sections.

4.8.1. Prognostic and Diagnostic Assays

The present methods provide means for determining if a subject has (diagnostic) or is at risk of developing (prognostic) a disease, condition or disorder that is associated with an aberrant htrb activity, e.g., an aberrant level of htrb protein or an aberrant htrb bioactivity. Examples of such diseases, conditions or disorders include without limitation: inflammatory diseases including rheumatoid arthritis, inflammatory bowel disorder, psoriasis, lupus erythematosus, ulcerative colitis and alopecia areata as well as diabetic nephropathy; cancers involving the growth factor cytokines or steroid hormone-responsive tumors (e.g. breast, prostate, or testicular cancer); vascular diseases or disorders (e.g. thrombotic stroke, ischemic stroke, as well as peripheral vascular disease resulting from atherosclerotic and thrombotic processes); cardiac disorders (e.g. myocardial infarction, unstable angina and ishemic heart disease); cardiovascular system diseases and disorders (e.g. those resulting from hypertension, hypotension, cardiomyocyte hypertrophy and congestive heart failure) wound healing; limb regeneration; neurological damage or disease (e.g. that associated with Alzheimer's disease, Parkinson's disease, AIDS-related complex, or cerebral palsy); or other diseases conditions or disorders which result from aberrations or alterations of htrb-dependent processes including: collateral growth and remodeling of cardiac blood vessels, angiogenesis, cellular transformation through autocrine or paracrine mechanisms, chemotactic stimulation of cells (e.g. endothelial), neurite outgrowth of neuronal precursor cell types (e.g. PC12 phaeochromoctoma), maintenance of neural physiology of mature neurons, proliferation of embryonic mesenchyme and limb-bud precursor tissue, mesoderm induction and other developmental processes, stimulation of collagenase and plasminogen activator secretion, tumor vascularization, as well as tumor invasion and metastasis.

Accordingly, the invention provides methods for determining whether a subject has or is likely to develop, a disease or condition that is caused by or contributed to by an abnormal htrb level or bioactivity, for example, comprising determining the level of an htrb gene or protein, an htrb bioactivity and/or the presence of a mutation or particular polymorphic variant in the htrb gene.

In one embodiment, the method comprises determining whether a subject has an abnormal mRNA and/or protein level of htrb, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the htrb protein or mRNA level is determined and compared to the level of htrb protein or mRNA level in a healthy subject. An abnormal level of htrb polypeptide or mRNA level is likely to be indicative of an aberrant htrb activity.

In another embodiment, the method comprises measuring at least one activity of htrb. For example, the affinity of htrb for heparin, can be determi+ned, e.g., as described herein. Similarly, the constant of affinity of an htrb protein of a subject with a binding partner (e.g. an IL-1 type I or type II receptor) can be determined. Comparison of the results obtained with results from similar analysis performed on htrb proteins from healthy subjects is indicative of whether a subject has an abnormal htrb activity.

In preferred embodiments, the methods for determining whether a subject has or is at risk for developing a disease, which is caused by or contributed to by an aberrant htrb activity is characterized as comprising detecting, in a sample of cells from the subject, the presence or absence of a genetic alteration characterized by at least one of (i) an alteration affecting the integrity of a gene encoding an htrb polypeptide, or (ii) the mis-expression of the htrb gene. For example, such genetic alterations can be detected by ascertaining the existence of at least one of (i) a deletion of one or more nucleotides from an htrb gene, (ii) an addition of one or more nucleotides to an htrb gene, (iii) a substitution of one or more nucleotides of an htrb gene, (iv) a gross chromosomal rearrangement of an htrb gene, (v) a gross alteration in the level of a messenger RNA transcript of an htrb gene, (vii) aberrant modification of an htrb gene, such as of the methylation pattern of the genomic DNA, (vii) the presence of a non-wild type splicing pattern of a messenger RNA transcript of an htrb gene, (viii) a non-wild type level of an htrb polypeptide, (ix) allelic loss of an htrb gene, and/or (x) inappropriate post-translational modification of an htrb polypeptide. As set out below, the present invention provides a large number of assay techniques for detecting alterations in an htrb gene. These methods include, but are not limited to, methods involving sequence analysis, Southern blot hybridization, restriction enzyme site mapping, and methods involving detection of absence of nucleotide pairing between the nucleic acid to be analyzed and a probe. These and other methods are further described infra.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene, such as a single nucleotide polymorphism (“SNP”), in a subject can render the subject susceptible to developing a specific disease or disorder. Polymorphic regions in genes, e.g, htrb genes, can be identified, by determining the nucleotide sequence of genes in populations of individuals. If a polymorphic region, e.g., SNP is identified, then the link with a specific disease can be determined by studying specific populations of individuals, e.g, individuals which developed a specific disease, such as congestive heart failure, hypertension, hypotension, or a cancer (e.g. a cancer involving growth of a steroid responsive tumor or tumors). A polymorphic region can be located in any region of a gene, e.g., exons, in coding or non coding regions of exons, introns, and promoter region.

It is likely that htrb genes comprise polymorphic regions, specific alleles of which may be associated with specific diseases or conditions or with an increased likelihood of developing such diseases or conditions. Thus, the invention provides methods for determining the identity of the allele or allelic variant of a polymorphic region of an htrb gene in a subject, to thereby determine whether the subject has or is at risk of developing a disease or disorder associated with a specific allelic variant of a polymorphic region.

In an exemplary embodiment, there is provided a nucleic acid composition comprising a nucleic acid probe including a region of nucleotide sequence which is capable of hybridizing to a sense or antisense sequence of an htrb gene or naturally occurring mutants thereof, or 5′ or 3′ flanking sequences or intronic sequences naturally associated with the subject htrb genes or naturally occurring mutants thereof. The nucleic acid of a cell is rendered accessible for hybridization, the probe is contacted with the nucleic acid of the sample, and the hybridization of the probe to the sample nucleic acid is detected. Such techniques can be used to detect alterations or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention, several probes capable of hybridizing specifically to allelic variants, such as single nucleotide polymorphisms, are attached to a solid phase support, e.g., a “chip”. Oligonucleotides can be bound to a solid support by a variety of processes, including lithography. For example a chip can hold up to 250,000 oligonucleotides. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In certain embodiments, detection of the alteration comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligase chain reaction (LCR) (see, e.g., Landegran et al. (1988) Science 241:1077-1080; and Nakazawa et al. (1994) PNAS 91:360-364), the latter of which can be particularly useful for detecting point mutations in the htrb gene (see Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to an htrb gene under conditions such that hybridization and amplification of the htrb gene (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In a preferred embodiment of the subject assay, mutations in, or allelic variants, of an htrb gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the htrb gene and detect mutations by comparing the sequence of the sample htrb with the corresponding wild-type (control) sequence. Exemplary sequencing reactions include those based on techniques developed by Maxim and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of“mismatch cleavage” starts by providing heteroduplexes formed by hybridizing (labelled) RNA or DNA containing the wild-type htrb sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in htrb cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an htrb sequence, e.g., a wild-type htrb sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations or the identity of the allelic variant of a polymorphic region in htrb genes. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control htrb nucleic acids are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting point mutations or the identity of the allelic variant of a polymorphic region include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) ?roc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of an htrb gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in an htrb gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent No. 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA TM in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer.J. Hum. Genet. 52:46-59 (1993)).

For mutations that produce premature termination of protein translation, the protein truncation test (PT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

The methods described herein may be performed, for example, by utilizing pre-packaged diagnostic kits comprising at least one probe nucleic acid, primer set; and/or antibody reagent described herein, which may be conveniently used, e.g., in clinical settings to diagnose patients exhibiting symptoms or family history of a disease or illness involving an htrb polypeptide.

Any cell type or tissue may be utilized in the diagnostics described below. In a preferred embodiment a bodily fluid, e.g., blood, is obtained from the subject to determine the presence of a mutation or the identity of the allelic variant of a polymorphic region of an htrb gene. A bodily fluid, e.g, blood, can be obtained by known techniques (e.g. venipuncture). Alternatively, nucleic acid tests can be performed on dry samples (e.g. hair or skin). For prenatal diagnosis, fetal nucleic acid samples can be obtained from maternal blood as described in International Patent Application No. WO91/07660 to Bianchi. Alternatively, amniocytes or chorionic villi may be obtained for performing prenatal testing.

When using RNA or protein to determine the presence of a mutation or of a specific allelic variant of a polymorphic region of an htrb gene, the cells or tissues that may be utilized must express the htrb gene. Preferred cells for use in these methods include cardiac cells (see Examples). Alternative cells or tissues that can be used, can be identified by determining the expression pattern of the specific htrb gene in a subject, such as by Northern blot analysis.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Antibodies directed against wild type or mutant htrb polypeptides or allelic variants thereof, which are discussed above, may also be used in disease diagnostics and prognostics. Such diagnostic methods, may be used to detect abnormalities in the level of htrb polypeptide expression, or abnormalities in the structure and/or tissue, cellular, or subcellular location of an htrb polypeptide. Structural differences may include, for example, differences in the size, electronegativity, or antigenicity of the mutant htrb polypeptide relative to the normal htrb polypeptide. Protein from the tissue or cell type to be analyzed may easily be detected or isolated using techniques which are well known to one of skill in the art, including but not limited to western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of htrb polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the htrb polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-htrb polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Inmunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a gene or gene product or polymorphic variants can be used to monitor the course of treatment or therapy.

4.8.2. Pharmacogenomics

Knowledge of the particular alteration or alterations, resulting in defective or deficient htrb genes or proteins in an individual (the htrb genetic profile), alone or in conjunction with information on other genetic defects contributing to the same disease (the genetic profile of the particular disease) allows a customization of the therapy for a particular disease to the individual's genetic profile, the goal of “pharmacogenomics”. For example, subjects having a specific allele of an htrb gene may or may not exhibit symptoms of a particular disease or be predisposed of developing symptoms of a particular disease. Further, if those subjects are symptomatic, they may or may not respond to a certain drug, e.g., a specific htrb therapeutic, but may respond to another. Thus, generation of an htrb genetic profile, (e.g., categorization of alterations in htrb genes which are associated with the development of a particular disease), from a population of subjects, who are symptomatic for a disease or condition that is caused by or contributed to by a defective and/or deficient htrb gene and/or protein (an htrb genetic population profile) and comparison of an individual's htrb profile to the population profile, permits the selection or design of drugs that are expected to be safe and efficacious for a particular patient or patient population (i.e., a group of patients having the same genetic alteration).

For example, an htrb population profile can be performed, by determining the htrb profile, e.g., the identity of htrb genes, in a patient population having a disease, which is caused by or contributed to by a defective or deficient htrb gene. Optionally, the htrb population profile can further include information relating to the response of the population to an htrb therapeutic, using any of a variety of methods, including, monitoring: 1) the severity of symptoms associated with the htrb related disease, 2) htrb gene expression level, 3) htrb mRNA level, and/or 4) htrb protein level. and (iii) dividing or categorizing the population based on the particular genetic alteration or alterations present in its htrb gene or an htrb pathway gene. The htrb genetic population profile can also, optionally, indicate those particular alterations in which the patient was either responsive or non-responsive to a particular therapeutic. This information or population profile, is then useful for predicting which individuals should respond to particular drugs, based on their individual htrb profile.

In a preferred embodiment, the htrb profile is a transcriptional or expression level profile and step (i) is comprised of determining the expression level of htrb proteins, alone or in conjunction with the expression level of other genes, known to contribute to the same disease. The htrb profile can be measured in many patients at various stages of the disease.

Pharmacogenomic studies can also be performed using transgenic animals. For example, one can produce transgenic mice, e.g., as described herein, which contain a specific allelic variant of an htrb gene. These mice can be created, e.g, by replacing their wild-type htrb gene with an allele of the human htrb gene. The response of these mice to specific htrb therapeutics can then be determined.

4.8.3. Monitoring of Effects of htrb Therapeutics During Clinical Trials

The ability to target populations expected to show the highest clinical benefit, based on the htrb or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of htrb as a marker is useful for optimizing effective dose).

The treatment of an individual with an htrb therapeutic can be monitored by determining htrb characteristics, such as htrb protein level or activity, htrb mRNA level, and/or htrb transcriptional level. This measurements will indicate whether the treatment is effective or whether it should be adjusted or optimized. Thus, htrb can be used as a marker for the efficacy of a drug during clinical trials.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of an htrb protein, mRNA, or genomic DNA in the preadministration sample; (iii) obtaining one or more post-administration samples from the subject; (iv) detecting the level of expression or activity of the htrb protein, mRNA, or genomic DNA in the post-administration samples; (v) comparing the level of expression or activity of the htrb protein, mRNA, or genomic DNA in the preadministration sample with the htrb protein, IRNA, or genomic DNA in the post administration sample or samples; and (vi) altering the administration of the agent to the subject accordingly. For example, increased administration of the agent may be desirable to increase the expression or activity of htrb to higher levels than detected, i.e., to increase the effectiveness of the agent. Alternatively, decreased administration of the agent may be desirable to decrease expression or activity of htrb to lower levels than detected, i.e., to decrease the effectiveness of the agent.

Cells of a subject may also be obtained before and after administration of an htrb therapeutic to detect the level of expression of genes other than htrb, to verify that the htrb therapeutic does not increase or decrease the expression of genes which could be deleterious. This can be done, e.g., by using the method of transcriptional profiling. Thus, mRNA from cells exposed in vivo to an htrb therapeutic and mRNA from the same type of cells that were not exposed to the htrb therapeutic could be reverse transcribed and hybridized to a chip containing DNA from numerous genes, to thereby compare the expression of genes in cells treated and not treated with an htrb-therapeutic. If, for example an htrb therapeutic turns on the expression of a proto-oncogene in an individual, use of this particular htrb therapeutic may be undesirable.

4.9. htrb Therapeutics and Methods of Treatment

The present invention provides for both prophylactic and therapeutic methods of treating a subject having or likely to develop a disorder associated with aberrant interleukin-1 expression or activity, e.g., inflammation or autoimmune disorders. The cytokines interleukin-1 (IL-1) and tumour necrosis factor (TNF) are important mediators of inflammatory responses, and appear to play a central role in the pathogenesis of many chronic inflammatory diseases. It is now well documented that their biological activities in vivo are sufficient to reproduce local inflammation and matrix catabolism by attracting and activating white blood cells to tissues, and stimulating their secretion of other lymphocytotropic cytokines and catabolic enzymes. Higher production of these cytokines have also been associated with response to infection, where local induction of IL-1 and TNF facilitates the elimination of the microbial invasion. Classic studies however also report that in some infectious conditions very high levels of monocytic cytokines are produced, which activate a cascade of concomitant events such as tissue catabolism, vascular reactivity and hyper-coagulation with damaging effects on the host.

For example, cytokines function throughout development and may be of particular importance in the development and function of the human placenta (reviewed in Jokhi et al. (1997) Cytokine 9: 126-37). A variety of cytokines have been demonstrated at the placental-uterine interface, but the exact cellular sources of production have not yet been identified due to the complex tissue topography of the implantation site. The expression of the cytokines EGF, interleukin 1 beta (IL-1 beta), IL-2, IL-3, interferon alpha (IFN-alpha), IFN-gamma, tumour necrosis factor alpha (TNF-alpha) and transforming growth factor beta 1 (TGF-beta 1) have been assayed from cells isolated from the placenta and decidua. Furthermore, the expression of the cytokine receptors IGF-1r, PDGF-r alpha/beta, IL-1rII, IL-6r, IL-7r, IFN-gamma r, TNF-rp80 and endoglin by placental and uterine cells has been assessed by both immunohistological and flow cytometric methods. These studies reveal a complex array of cytokine activities at the human placental-uterine interface.

The pro-inflammatory cytokines IL-1, IL-6 and tumor necrosis factor-alpha (TNFa) appear to function in the link between prenatal intrauterine infection (IUI) and neonatal brain damage. Furthermore, maternal IUI increases the risk of preterm delivery, which in turn is associated with an increased risk of intraventricular hemorrhage, neonatal white matter damage, and subsequent cerebal palsy (Dammann et al. (1997) Pediatr Res 42: 1-8). IL-1, IL-6, and TNFa have been found associated with IUI, preterm birth, neonatal infections, and neonatal brain damage. The presence of such cytokines in the three relevant maternal/fetal compartments (uterus, fetal circulation, and fetal brain) and their potential ability of the cytokines to cross boundaries (both placental and the blood-brain barrier) between these compartments suggests their potential role in intraventricular hemorrhage, neonatal white matter damage during prenatal maternal infection. Therefore interrupting the proinflammatory cytokine cascade mediated by IL-1 might prevent later disability in those born near the end of the second trimester.

Interleukin-1 beta (IL-1 β) is present in normal amniotic fluid and is produced by human placental macrophages. The amount of IL-1 β detected in the second trimester amniotic fluid has been shown to exhibit a threefold increase with the onset of labor. IL-1 β is a potent stimulator of the synthesis of prostaglandins by decidua and by amnion. High levels of the prostaglandins PGE₂ and PGF2α in the amniotic fluid have been associated with preterm labor and intraamniotic infection. This may be explained by the fact that amnion from women with preterm labor and histologic chorioamnionitis produced more PGE₂ than amnion from women without placental inflammation. Such elevated levels of PGE₂ have been associated with premature low birth weight (PBLW) even in the absence of clinical or subclinical genitourinary tract infection and indeed the majority of PLBW deliveries may be caused by an infection of unknown origin. IL-1 was the first cytokine implicated in the onset of labor in the presence of infection. IL-1 is produced in vitro by human decidua in response to bacterial products. In patients with preterm labor and bacteria in the amniotic cavity, amniotic fluid IL-1 bioactivity and concentrations are elevated. Placental necrosis and fetal resorption can be induced in rats by the injection of recombinant human IL-1 β on day 12 of gestation. Furthermore, both the amniotic fluid IL-1 β concentration and bioactivity are elevated during labor compared to controls. In addition, IL-1 β is known to stimulate prostaglandin production by amnion and decidua in vitro.

Accordingly, htrb therapeutics of the present invention include those which antagonize interleukin-1 dependent disorders of the human placental including intraventricular hemorrhage, neonatal white matter damage and subsequent cerebal palsy, and the occurrence of premature low birth weight deliveries.

4.9.1. Prophylactic Methods

In one aspect, the invention provides a method for preventing in a subject, a disease or condition associated with an aberrant htrb expression or activity by administering to the subject an agent which modulates htrb expression or at least one htrb activity. Subjects at risk for such a disease can be identified by a diagnostic or prognostic assay, e.g., as described herein. Administration of a prophylactic agent can occur prior to the manifestation of symptoms characteristic of the htrb aberrancy, such that a disease or disorder is prevented or, alternatively, delayed in its progression. Depending on the type of htrb aberrancy, for example, a htrb agonist or htrb antagonist agent can be used for treating the subject prophylactically. The prophylactic methods are similar to therapeutic methods of the present invention and are further discussed in the following subsections.

4.9.2. Therapeutic Methods

In general, the invention provides methods for treating a disease or condition which is caused by or contributed to by an aberrant htrb activity comprising administering to the subject an effective amount of a compound which is capable of modulating an htrb activity. Among the approaches which may be used to ameliorate disease symptoms involving an aberrant htrb activity are, for example, antisense, ribozyme, and triple helix molecules described above. Examples of suitable compounds include the antagonists, agonists or homologues described in detail herein.

4.9.3. Effective Dose

Toxicity and therapeutic efficacy of such compounds can be determined by standard pharmaceutical procedures in cell cultures or experimental animals, e.g., for determining The Ld50 (The Dose Lethal To 50% Of The Population) And The Ed50 (the dose therapeutically effective in 50% of the population). The dose ratio between toxic and therapeutic effects is the therapeutic index and it can be expressed as the ratio LD50/ED50. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

4.9.4. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such, as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possiblity of local noninvasive delivery of drugs over an extended period of time. This technology utilizes microspheres of precapillary size which can be injected via a coronary chatheter into any selected part of the e.g. heart or other organs without causing inflammation or ischemia The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives, in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a gene delivery system for the therapeutic htrb gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). An htrb gene, such as any one of the sequences represented in the group consisting of SEQ ID Nos. 1 or 3 or the htrb alternative 5′ ends or a sequence homologous thereto can be delivered in a gene therapy construct by electroporation using techniques described, for example, by Dev et al. ((1994) Cancer Treat Rev 20:105-115).

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

4.10. Kits

The invention further provides kits for use in diagnostics or prognostic methods or for treating a disease or condition associated with an aberrant htrb protein. The invention also provides kits for determining which htrb therapeutic should be administered to a subject. The invention encompasses kits for detecting the presence of htrb mRNA or protein in a biological sample or for determining the presence of mutations or the identity of polymorphic regions in an htrb gene. For example, the kit can comprise a labeled compound or agent capable of detecting htrb protein or mRNA in a biological sample; means for determining the amount of htrb in the sample; and means for comparing the amount of htrb in the sample with a standard. The compound or agent can be packaged in a suitable container. The kit can further comprise instructions for using the kit to detect htrb mRNA or protein.

In one embodiment, the kit comprises a pharmaceutical composition containing an effective amount of an htrb antagonist therapeutic and instruction for use in treating or preventing hypertension. In another embodiment, the kit comprises a pharmaceutical composition comprising an effective amount of an htrb agonist therapeutic and instructions for use in treating insect bites. Generally, the kit comprises a pharmaceutical composition comprising an effective amount of an htrb agonist or antagonist therapeutic and instructions for use as an analgesic. For example, the kit can comprise a pharmaceutical composition comprising an effective amount of an htrb agonist therapeutic and instructions for use as an analgesic.

Yet other kits can be used to determine whether a subject has or is likely to develop a disease or condition associated with an aberrant htrb activity. Such a kit can comprise, e.g., one or more nucleic acid probes capable of hybridizing specifically to at least a portion of an htrb gene or allelic variant thereof, or mutated form thereof

4.11. Additional Uses for htrb Proteins and Nucleic Acids

The htrb nucleic acids of the invention can further be used in the following assays. In one embodiment, the human htrb nucleic acid having SEQ ID No. 1 or a portion thereof, or a nucleic acid which hybridizes thereto can be used to determine the precise chromosomal localization of an htrb gene within the IL-1 locus. Furthermore, the htrb gene can also be used as a chromosomal marker in genetic linkage studies involving genes other than htrb.

Chromosomal localization of a gene can be performed by several methods well known in the art. For example, Southern blot hybridization or PCR mapping of somatic cell hybrids can be used for determining on which chromosome or chromosome fragment a specific gene is located. Other mapping strategies that can similarly be used to localize a gene to a chromosome or chromosomal region include in situ hybridization, prescreening with labeled flow-sorted chromosomes and preselection by hybridization to construct chromosome specific-cDNA libraries.

Furthermore, fluorescence in situ hybridization (FISH) of a nucleic acid, e.g., an htrb nucleic acid, to a metaphase chromosomal spread is a one step method that provides a precise chromosomal location of the nucleic acid. This technique can be used with nucleic acids as short as 500 or 600 bases; however, clones larger than 2,000 bp have a higher likelihood of binding to a unique chromosomal location with sufficient signal intensity for simple detection. Such techniques are described, e.g, in Verma et al., Human Chromosomes: a Manual of Basic Techniques, Pergamon Press, New York (1988). Using such techniques, a gene can be localized to a chromosomal region containing from about 50 to about 500 genes.

If the htrb gene is shown to be localized in a chromosomal region which cosegregates, i.e., which is associated, with a specific disease, the differences in the cDNA or genomic sequence between affected and unaffected individuals are determined. The presence of a mutation in some or all of the affected individuals but not in any normal individuals, will be indicative that the mutation is likely to be causing or contributing to the disease.

The present invention is further illustrated by the following examples which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, ₂nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Acadernic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

4.12 Detecting Gene Expression and Microarray Transcription Profiling

The invention provides for iterative methodologies for determining inflammatory gene regulatory pathways by: (1) cloning a first gene regulating a given inflammatory gene; (2) using that first inflammatory regulatory gene in an expression profiling procedure to isolate other inflammatory pathway genes whose expression is altered by over or under-expression of the first gene; and (3) still further determining the expression profile of a cell over or under expressing each of the other inflammatory pathway genes to isolate still other inflammatory pathway genes whose expression is altered by over or under-expression of each of the “other” inflammatory pathway genes. Many different methods are known in the art for measuring gene expression. Classical methods include quantitative RT-PCR, Northern blots and ribonuclease protection assays. Such methods may be used to examine expression of individual genes as well as entire gene clusters. However, as the number of genes to be examined increases, the time and expense may become prohibitive.

Large scale detection methods allow faster, less expensive analysis of the expression levels of many genes simultaneously. Such methods typically involve an ordered array of probes affixed to a solid substrate. Each probe is capable of hybridizing to a different set of nucleic acids. In one method, probes are generated by amplifying or synthesizing a substantial portion of the coding regions of various genes of interest. These genes are then spotted onto a solid support. mRNA samples are obtained, converted to cDNA, amplified and labeled (usually with a fluorescence label). The labeled cDNAs are then applied to the array, and cDNAs hybridize to their respective probes in a manner that is linearly related to their concentration. Detection of the label allows measurement of the amount of each cDNA adhered to the array.

Many methods for performing such DNA array experiments are well known in the art. Exemplary methods are described below but are not intended to be limiting.

Arrays are often divided into microarrays and macroarrays, where microarrays have a much higher density of individual probe species per area. Microarrays may have as many as 1000 or more different probes in a 1 cm² area. There is no concrete cut-off to demarcate the difference between micro- and macroarrays, and both types of arrays are contemplated for use with the invention. However, because of their small size, microarrays provide great advantages in speed, automation and cost-effectiveness.

Microarrays are known in the art and consist of a surface to which probes that correspond in sequence to gene products (e.g., cDNAs, mRNAs, oligonucleotides) are bound at known positions. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least 100 genes and more preferably, 500, 1000, 4000 or more. In certain embodiments, the most preferred arrays will have about 98-100% of the genes of a particular organism represented. In other embodiments, the invention provides customized microarrays that have binding sites corresponding to fewer, specifically selected genes. Microarrays with fewer binding sites are cheaper, smaller and easier to produce. In particular, the invention provides microarrays customized for the determination of graft status. In preferred embodiments customized microarrays comprise binding sites for fewer than 4000, fewer than 1000, fewer than 200 or fewer than 50 genes, and comprise binding sites for at least 2, preferably at least 3, 4, 5 or more genes of any of clusters A, B, C, D, E, F or G. Preferably, the microarray has binding sites for genes relevant to testing and confirming a biological network model of interest. Several exemplary human microarrays are publically available. The Affymetrix GeneChip HUM 6.8K is an oligonucleotide array composed of 7,070 genes. A microarray with 8,150 human cDNAs was developed and published by Research Genetics (Bittner et al., 2000, Nature 406:443-546).

The probes to be affixed to the arrays are typically polynucleotides. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo p1 version 5.0 (National Biosciences). In the case of binding sites corresponding to very long genes, it will sometimes be desirable to amplify segments near the 3′ end of the gene so that when oligo-dT primed CDNA probes are hybridized to the microarray, less-than-full length probes will bind efficiently. Random oligo-dT priming may also be used to obtain cDNAs corresponding to as yet unknown genes, known as ESTs. Certain arrays use many small oligonucleotides corresponding to overlapping portions of genes. Such oligonucleotides may be chemically synthesized by a variety of well known methods. Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., 1993, PNA hybridizes to complementary oligonucleotides obeying the Watson-Crick hydrogen-bonding rules, Nature 365:566-568; see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., 1995, Differential gene expression in the murine thymus assayed by quantitative hybridization of arrayed cDNA clones, Genomics 29:207-209). In yet another embodiment, the polynucleotide of the binding sites is RNA.

The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Science 270:467-470. This method is especially useful for preparing microarrays of cDNA. (See also DeRisi et al., 1996, Nature Genetics 14:457-460; Shalon et al., 1996, Genome Res. 6:639-645; and Schena et al., 1995, Proc. Natl. Acad. Sci. USA 93:10539-11286). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, 11: 687-90). When these methods are used, oligonucleotides of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

The nucleic acids to be contacted with the microarray may be prepared in a variety of ways. Methods for preparing total and poly(A)+RNA are well known and are described generally in Sambrook et al., supra. Labeled cDNA is prepared from mRNA by oligo dT-primed or random-primed reverse transcription, both of which are well known in the art (see e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Nature Biotech. 14:1675). The cDNAs or RNAs can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescent labels are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Academic Press San Diego, Calif.).

In another embodiment, a label other than a fluorescent label is used. For example, a radioactive label, or a pair of radioactive labels with distinct emission spectra, can be used (see Zhao et al., 1995, Gene 156:207; Pietu et al., 1996, Genome Res. 6:492). However, use of radioisotopes is a less-preferred embodiment.

Nucleic acid hybridization and wash conditions are chosen so that the population of labeled nucleic acids will specifically hybridize to appropriate, complementary nucleic acids affixed to the matrix. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches).

Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array can be, preferably, detected by scanning confocal laser microscopy. When two fluorophores are used, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously. Fluorescent microarray scanners are commercially available from Affymetrix, Packard BioChip Technologies, BioRobotics and many other suppliers.

Signals are recorded, quantitated and analyzed using a variety of computer software. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

According to the method of the invention, the relative abundance of an mRNA in two samples is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In one embodiment of the invention, transcript arrays reflecting the transcriptional state of a cell of interest are made by hybridizing a mixture of two differently labeled sets of cDNAs, to the microarray. One cell is a cell of interest, while the other is used as a standardizing control. The relative hybridization of each cell's cDNA to the microarray then reflects the relative expression of each gene in the two cell. For example, to assess gene expression in a variety of breast cancers, Perou et al. (2000, supra) hybridized fluorescently-labeled cDNA from each tumor to a microarray in conjunction with a standard mix of cDNAs obtained from a set of breast cancer cell lines. In this way, gene expression in each tumor sample was compared against the same standard, permitting easy comparisons between tumor samples.

In preferred embodiments, the data obtained from such experiments reflects the relative expression of each gene represented in the microarray. Expression levels in different samples and conditions may be compared using a variety of statistical methods.

A variety of statistical methods are available to assess the degree of relatedness in expression patterns of different genes. The statistical methods may be broken into two related portions: metrics for determining the relatedness of the expression pattern of one or more gene, and clustering methods, for organizing and classifying expression data based on a suitable metric (Sherlock, 2000, Curr. Opin. Immunol. 12:201-205; Butte et al., 2000, Pacific Symposium on Biocomputing, Hawaii, World Scientific, p.418-29).

In one embodiment, Pearson correlation may be used as a metric. In brief, for a given gene, each data point of gene expression level defines a vector describing the deviation of the gene expression from the overall mean of gene expression level for that gene across all conditions. Each gene's expression pattern can then be viewed as a series of positive and negative vectors. A Pearson correlation coefficient can then be calculated by comparing the vectors of each gene to each other. An example of such a method is described in Eisen et al. (1998, supra). Pearson correlation coefficients account for the direction of the vectors, but not the magnitudes.

In another embodiment, Euclidean distance measurements may be used as a metric. In these methods, vectors are calculated for each gene in each condition and compared on the basis of the absolute distance in multidimensional space between the points described by the vectors for the gene.

In a further embodiment, the relatedness of gene expression patterns may be determined by entropic calculations (Butte et al. 2000, supra). Entropy is calculated for each gene's expression pattern. The calculated entropy for two genes is then compared to determine the mutual information. Mutual information is calculated by subtracting the entropy of the joint gene expression patterns from the entropy for calculated for each gene individually. The more different two gene expression patterns are, the higher the joint entropy will be and the lower the calculated mutual information. Therefore, high mutual information indicates a non-random relatedness between the two expression patterns.

The different metrics for relatedness may be used in various ways to identify clusters of genes. In one embodiment, comprehensive pairwise comparisons of entropic measurements will identify clusters of genes with particularly high mutual information. In preferred embodiments, expression patterns for two genes are correlated if the normalized mutual information score is greater than or equal to 0.7, and preferably greater than 0.8, greater than 0.9 or greater than 0.95. In alternative embodiments, a statistical significance for mutual information may be obtained by randomly permuting the expression measurements 30 times and determining the highest mutual information measurement obtained from such random associations. All clusters with a mutual information higher than can be obtained randomly after 30 permutations are statistically significant. In a further embodiment, expression patterns for two genes are correlated if the correlation coefficient is greater than or equal to 0.8, and preferably greater than 0.85, 0.9 or, most preferably greater than 0.95.

In another embodiment, agglomerative clustering methods may be used to identify gene clusters. In one embodiment, Pearson correlation coefficients or Euclidean metrics are determined for each gene and then used as a basis for forming a dendrogram. In one example, genes were scanned for pairs of genes with the closest correlation coefficient. These genes are then placed on two branches of a dendrogram connected by a node, with the distance between the depth of the branches proportional to the degree of correlation. This process continues, progressively adding branches to the tree. Ultimately a tree is formed in which genes connected by short branches represent clusters, while genes connected by longer branches represent genes that are not clustered together. The points in multidimensional space by Euclidean metrics may also be used to generate dendrograms.

In yet another embodiment, divisive clustering methods may be used. For example, vectors are assigned to each gene's expression pattern, and two random vectors are generated. Each gene is then assigned to one of the two random vectors on the basis of probability of matching that vector. The random vectors are iteratively recalculated to generate two centroids that split the genes into two groups. This split forms the major branch at the bottom of a dendrogram. Each group is then further split in the same manner, ultimately yielding a fully branched dendrogram.

In a further embodiment, self-organizing maps (SOM) may be used to generate clusters. In general, the gene expression patterns are plotted in n-dimensional space, using a metric such as the Euclidean metrics described above. A grid of centroids is then placed onto the n-dimensional space and the centroids are allowed to migrate towards clusters of points, representing clusters of gene expression. Finally the centroids represent a gene expression pattern that is a sort of average of a gene cluster. In certain embodiments, SOM may be used to generate centroids, and the genes clustered at each centroid may be further represented by a dendrogram. An exemplary method is described in Tamayo et al., 1999, PNAS 96:2907-12. Once centroids are formed, correlation must be evaluated by one of the methods described supra.

In another aspect, the invention provides probe sets. Preferred probe sets are designed to detect expression of multiple genes and provide information about the status of a graft. Preferred probe sets of the invention comprise probes that are useful for the detection of at least two genes belonging to gene clusters A, B, C, D, E, F or G. Particularly preferred probe sets will comprise probes useful for the detection of at least three, at least four or at least five genes belonging to gene clusters A, B, C, D, E, F or G. Certain probe sets may additionally comprise probes that are useful for the detection of one or more genes of gene cluster H. Probe sets of the invention do not comprise probes useful for the detection of more than 10,000 gene transcripts, and preferred probe sets will comprise probes useful for the detection of fewer than 4000, fewer than 1000, fewer than 200, and most preferably fewer than 50 gene transcripts. Probe sets of the invention are particularly useful because they are smaller and cheaper than probe sets that are intended to detect as many genes as possible in a particular genome. The probe sets of the invention are targeted at the detection of gene transcripts that are informative about transplant status. Probe sets of the invention may comprise a large or small number of probes that detect gene transcripts that are not informative about transplant status. Such probes are useful as controls and for normalization. Probe sets may be a dry mixture or a mixture in solution. In preferred embodiments, probe sets of the invention are affixed to a solid substrate to form an array of probes. It is anticipated that probe sets may also be useful for multiplex PCR.

### 5. EXAMPLES

### Example 1:

### Cloning and Identification of Inflammatory Pathway Genes

In order to identify novel genes involved in proinflammatory cytokine signaling, we have developed an expression screen to isolate gene products having the capacity to modulate the activity of a cytokine responsive reporter (Kiss-Toth, E. et al. (2000) Journal of Immunological Methods 239:125-135). The promoter of the IL-8 chemokine gene regulating the expression of the firefly luciferase gene was used as a readout. The promoter fragment (−79 to +45: pIL8) contains an AP-1, a C/EBP and an NFκB site, a typical structure for inflammatory signal responsive promoters. We have also found that the level of secretion of human IL-8 into the medium in this system is correlated with the activity of the reporter systems; thus the reporters are a valid surrogate measure of the activity of the endogenous gene. We have isolated both repressors and activators of signaling and have derived a lower bound estimate of the number of gene products that play a role in controlling transcription of the IL-8 gene.

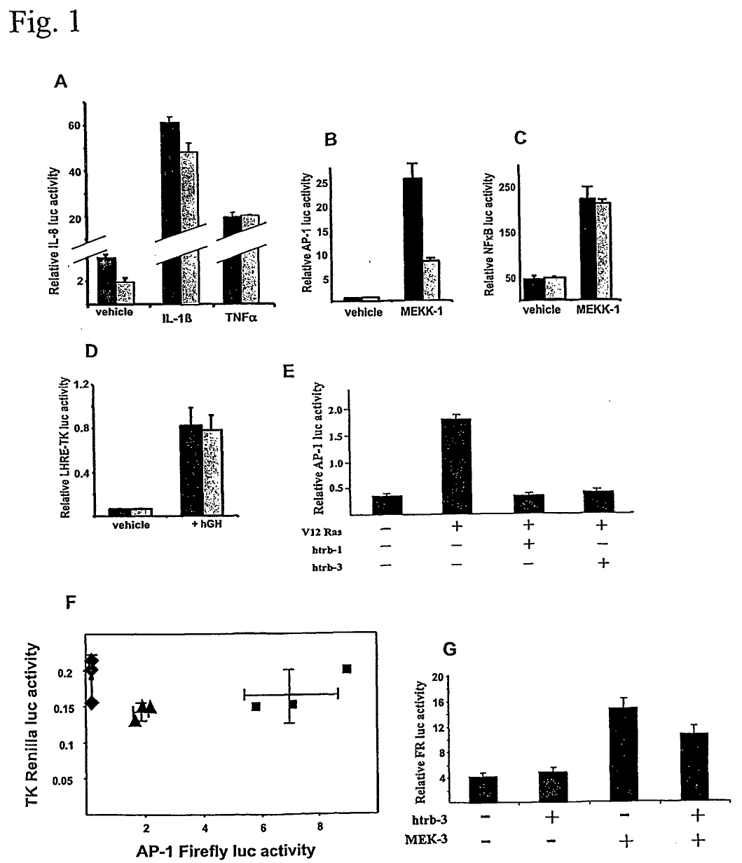

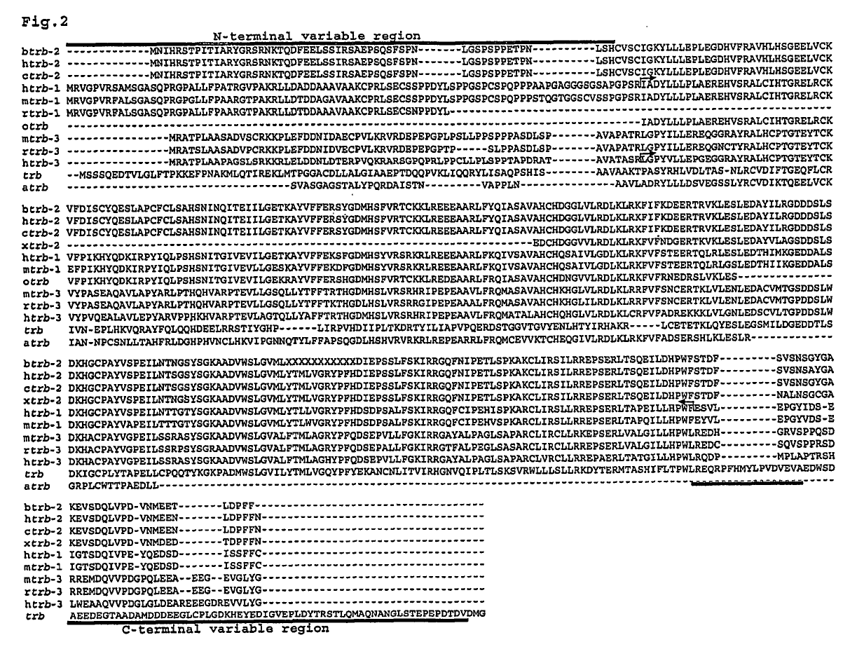

30 pools of ca 300 clones from an oligo-dT primed human peripheral blood mononuclear cell cDNA expression library in pCDM8 were screened in vivo against the reporter pIL-8—firefly luciferase with an internal control pTK (HSV Thymidine Kinase promoter)—Renilla luciferase, using a dual luciferase assay in HeLa cells. On pool (p) was positive, with a markedly elevated ratio of pIL-8/pTK activity compared to the other 29. We analyzed this and one negative pool in a single cell pIL-8-EGFP transcription assay, as described previously. The positive pool was active (FIG. 1B), with 5-10% of the cells in the transfected population showed induction of the reporter, compatible with the number expected to be productively transfected with one of the 300 clones in the pool. After pool breakdown, sequencing revealed that the active insert contained the entire coding region (ORF) for interferon-γ, in the sense orientation. This finding is compatible with reports that IFN-γ activates NF-κB. Further, despite the expected secretion of the cytokine, no paracrine “spreading” of the signal was seen in the GFP assay, and those cells that express it are apoptotic as judged by the fact that they are a small and bright and annexin V positive in two colour confocal fluorescence micrographs (data not shown). This is compatible with previous findings that IFN-γ can cause apoptosis, via up-regulation of FasL/Fas or Trail/Trail-R and that it induces NFκB activation, possibly synergistically after induction of TNF/TNFR family members.

The data from the screen provide a lower limit estimate for the number of genes involved in controlling pIL-8 luc function. The hit rate is 1/8000 and the library contains approximately 5×10⁶ independent clones, suggesting that the screen will detect about 600 clones encoding inhibitors or activators of pIL-8 luc transcription. The library is non-directional and oligo dT primed, with an average insert size of 2-3 kb. Clearly half the clones will be antisense and many will contain only 3′ UTRs. It is difficult to estimate what fraction of the clones n the library contain inserts encoding expressible proteins and indeed our results (see below) suggest that a full length coding region is not always required for the detection of relevant clones. Screening of a random primed human B-cell line (CB23) library for type II IL-1 receptor yielded an estimate of 1/300,000 for clones with a full length ORF. The frequency in our library is likely to be lower since it was prepared from a complex cell population, activated peripheral blood mononuclear cells, rather than a cell line and is oligo dT primed. Given this, and bearing in mind the SKIP result (see below), we estimate no more than 5 expressible clones for a rare mRNA (<1/25,000 in an clonal population), suggesting that a minimum of 100 distinct gene products, detectable by overexpression, play a role in regulating the IL-8 promoter. This number is reasonable, given the estimate of 40 genes on chromosome 3 in D. melanogaster, involved in controlling Dif and Dorsal translocation in fat body cells (see Wu & Anderson (1998) Nature 392: 93-7). This is likely only a subset of all components, since not all relevant gene products may alter activity when overexpressed. Indeed, we have found that IL-1RI and TLR4 do not activate pIL8, when over expressed in HeLa cells, unless exogenous ligand is added to the cultures.

Two of the clones obtained were active in altering levels of IL-8 —one as an activator and the other as an inhibitor of IL-8 mediated transcription. The activator was a full length Re1A cDNA clone in the sense orientation; an expected finding given the structure of the IL-8 promoter. The inhibitor cDNA clone encodes NAK-1, the human homolog of the murine orphan steroid hormone receptor Nur77, in the sense orientation. Nur77/N10/NAK-1/NGFI-B (a member of the NR4 subfamily) was previously identified as an immediate-early gene induced by a variety of stimuli in PC12 cells and fibroblasts [it is expressed in many cell types after activation by mitogens, for example]. A large body of literature implicates Nur77 and the closely related transcription factors NOR-1 and Nurr1 as playing a central role in apoptosis in many cell types, for example in T cell activation induced cell death (AICD) negative selection. Nur77/N10 transgenic mice show a dramatic reduction in both double and single positive thymocytes due to extensive early onset apoptosis. A recent report shows that Nur77 is one of a limited set of immediate early genes up-regulated (1 hour post-induction) in response to hen egg lysozyme stimulation of naive Ig^{HEL} B lymphocytes.

Transient overexpression of NAK-1 inhibited both the basal and IL-1 stimulated activity of the IL-8 promoter. In addition, stimulation of AP-1, NFκB and CRE reporters was suppressed by NAK-1. It also blocked activation of a synthetic LHRE promoter. Thus the inhibitory activity of NAK-1 is broad, possibly a consequence of overexpression. However, it is not a general inhibitor of transcription or translation, since the activity of the internal control reporter was not inhibited. Nur77 has been shown to facilitate AICD by a mechanism, which is only partially understood, and our data suggest that NAK-1 can act as an inhibitor of cytokine signaling. Steroid hormone receptors can block transactivation by NFκB and the activity of Nur77/NAK-1 can be potentiated by heterodimerisation with Nor-1, Nurrl and RXRs. Retinoids have also been shown to block cytokine gene expression by acting through NFκB. Our data suggest that NAK-1/Nur77 may be pro-apoptotic due in part in part to blockade of survival signals such as NFκB.

A second repressor clone encoded a 3′ UTR fragment in the sense orientation terminating in the poly-A tract. The sequence of the corresponding full-length transcript was deduced by searching human genomic and cDNA sequence databases. The partial sequence has been previously reported (GenBank AJ000480). The full length cDNA sequence was confirmed by sequencing ests obtained from the IMAGE collection. We have termed the gene product Stress Kinase Inhibitory Protein-1 (SKIP-1).

Screening pools from the cDNA expression library made from PBMC (Hamann, J. et al. (1993) Journal of Immunology 150:4920-27) further led us to identify a cDNA of previously unknown function. A homologous Drosophila gene, tribbles, has been described recently (Grosshans, J. et al. (2000) Cell 101:523-31; Seher, T. C. et al. (2000) Curr. Biol. 10:623-29; Mata, J. et al. (2000) Cell 101:511-22). Therefore we suggest the name human tribbles homologue-1 (htrb-1) for the isolated mammalian protein encoded by our clone.

### Example 2

### Analysis of htrb-1 Repression Specificity

FIG. 1 A show the effect of htrb-1 overexpression on IL-8 reporter activity. HeLa cells were transfected with the IL-8 luciferase reporter with or without a htrb-1 expression construct. Black bars indicate reporter activation without htrb-1 cotransfection, while gray bars show the reporter activation in the presence of 50 ng htrb-1 expression construct. Cells were stimulated with proinflammatory cytokines and the reporter activity was determined. B-D htrb-1 is a specific inhibitor of stress kinase signalling. HeLa cells were transiently transfected with AP-1 (B), NFkB (C) and human growth hormone (D) signalling pathway specific transcription reporters and were activated in the presence (gray bars) or absence (black bars) of htrb-1 expression plasmid by a MEKK-1 expression construct (B, C) or human growth hormone (D). E pAP-1 luc was stimulated with V12 Ras expression plasmid and the effect of htrb-1 and htrb-3 co-expression was investigated. F AP-1 reporter (diamonds) was stimulated with MEKK-1 expression plasmid (squares) and the effect of htrb-1 overexpression (triangles) on the stimulation was studied. The activity of constitutively active TK-Rluc versus the inducible AP-1 luc was plotted. G The p38 responsive pFR-luc+pFA-CHOP reporter was stimulated with MEK-3 expression construct. htrb-3 was co-transfected with the reporter in the presence or absence of the stimulus, as indicated.

Upon overexpression, htrb-1 was found to inhibit the basal activity of the IL-8 reporter, while the induction of this promoter by IL-1 and TNFalpha was not affected (FIG. 1A). The IL-8 promoter fragment used in this assay contains binding sites for NFkB and AP-1 and most of the cytokine inducible activity is mediated through to the NFkB site, while the AP-1 site contributes significantly to the basal promoter activity (Mukaida, N. et al. (1994) Journal of Leukocyte Biology 56:554-58). To determine which signaling pathway is inhibited by htrb-1, a cDNA encoding the full length htrb-1 ORF was tested against transcription reporters containing multiple copies of NFkB or AP-1 binding sites. The reporters were activated by MEKK-1. Overexpression of the htrb-1 protein suppressed the activation of the AP-1 but not of the NFkB reporter (FIGS. 1B and C) nor of a JAK/STAT pathway specific promoter (FIG. 1D). These data suggest that the observed negative effect of htrb-1 on signaling is specific for stress kinase pathways.

To confirm that the observed AP-1 inhibition is not restricted to MEKK-1 overexpression, V12 Ras was co-transfected with the AP-1 reporter in the absence or presence of htrb-1 or htrb-3 expression constructs (see below) (FIG. 1E). Ras-mediated activation was also blocked by elevated htrb levels. The effect of htrb overexpression on the activity of the constitutive HSV-TK promoter (FIG. 1F) and the possible involvement in activation of p38 mediated general stress responses (FIG. 1G) were assessed. Our data show that the effect of htrb-1 on the inhibition of MEKK-1 mediated AP-1 activation was specific for the inducible reporter, while the activity of the TK-control reporter was not affected by the treatments (FIG. 1F).

### Example 3

### Identification and Analysis of other htrb Genes

FIG. 2 shows multiple alignment of htrb protein family. Searching public DNA databases revealed 3 human htrb genes and further est-s, encoding trb homologs in vertebrates and insects. Putative protein sequences of human htrb family and the identified trb homologs have been aligned by ClustalX, using default parameters. Positions of htrb mutations are indicated by arrows. Abbreviations: h- Homo sapiens, m- Mus musculus, r- Rattus norvegicus, c- Canis canis, b- Bos taurus, x- Xenopus laevis, o- Oncorhynchus mykiss, a- Aedes aegypti.

Public database searching with the htrb-1 sequence identified two further human genes, htrb-2 and htrb-3, 41 and 51% identical to htrb-1 respectively. In addition, several putative trb gene products were found in vertebrate species and in insects (FIG. 2). All these proteins share a central similar kinase-like domain. In addition, each possesses short N-terminal (approx. 70-100 residues) and a C-terminal (approx. 25 residues) domains which are neither closely related to any other sequence in the databases, nor to each other. A partial human htrb-1 sequence has been reported (Wilkin, F. et al. (1997) European Journal of Biochemistry 248:660-68). Canine trb-2 has been described to be a highly labile cytoplasmic phosphoprotein lacking kinase activity: the mRNA is up-regulated by mitogens (Wilkin, F. et al. (1997) European Journal of Biochemistry 248:660-68; Wilkin, F. et al. (1996) Journal of Biological Chemistry 271:28451-57). A rat homologue of htrb-3 has been identified as a novel kinase-like gene induced during neuronal cell death (Mayumi-Matsuda, K. et al. (1999) Biochemical and Biophysical Research Communications 258:260-64). A Drosophila homologue is tribbles, which regulates mitosis and morphogenesis by regulating string/CDC25 (Grosshans, J. et al. (2000) Cell 101:523-31; Seher, T. C. et al. Curr. Biol. 10:623-29; Mata, J. et al. (2000) Cell 101:511-22; Rorth, P. et al. (2000) Mol. Cell 6:23-30). The trb kinase-like domain shows homology to protein serine-threonine kinases in general, and to calcium calmodulin kinases and SNF1 kinases in particular. However the trbs lack the active site lysine, and are predicted to be kinase dead, as shown for Canine trb-2 (Wilkin, F. et al. (1997) European Journal of Biochemistry 248:660-68) and tribbles (Grosshans, J. et al. (2000) Cell 101:523-31).

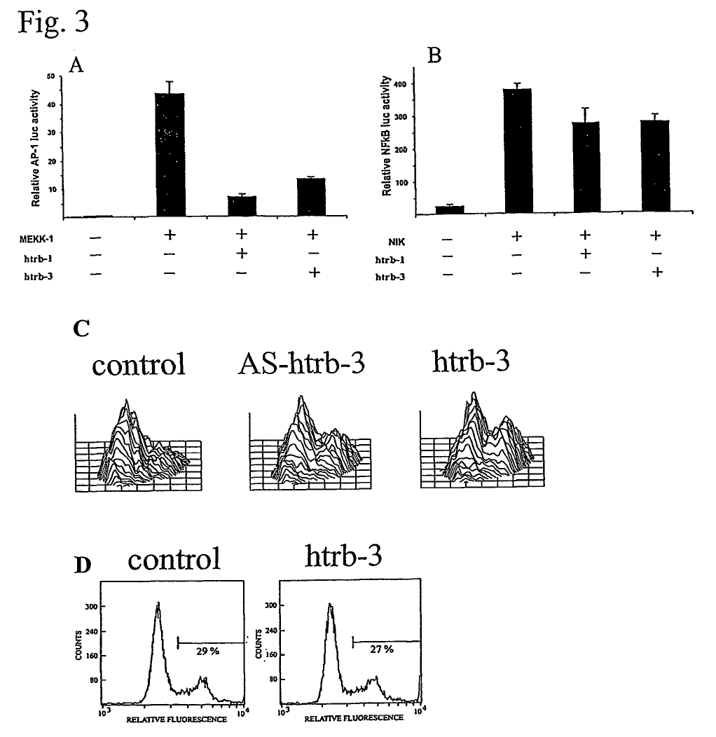

To determine whether htrb-3 has transcriptional regulatory activities similar to those of htrb-1, the effect of htrb-3 expression on activation of the AP-1 reporter was investigated. The two mammalian homologs had similar activities (see FIGS. 3A) and, furthermore, the observed AP-1 inhibition was not restricted to MEKK-1 overexpression, because V12 Ras mediated AP-1 activation was similarly affected by the expression of htrb-1 or htrb-3 expression constructs (FIG. 1E). In addition, the basal activity of a p38 responsive reporter was not influenced by htrb-3 and the activation of these kinases was slightly inhibited by overexpressing htrb-3 (FIG. 1G).

To further investigate the effect of htrb expression levels on stress kinase signalling, an antisense construct, expressing the htrb-1 or htrb-3 5′UTR and the region corresponding to the N-terminal variable region in reverse orientation was co-transfected into HeLa cells with AP-1 or NFkB reporters and a MEKK-1 or NIK expression construct as an activator (FIG. 3A, B). As observed with the full length sense cDNA, these constructs strongly inhibited AP-1 activation while having little effect on NFkB.

FIG. 3 shows expression of antisense htrb-1 and htrb-3 RNA inhibits stress kinase activation. Effects of cotransfected antisense htrb-1 or htrb-3 construct were measured on activation of AP-1 (A) and NFkB responsive reporters (B) by MEKK-1 or NIK expression plasmids, respectively. (C) HeLa cells were transfected with sense or antisense htrb-3 expression constructs, the cell size was measured by flow cytometry and the forward scatter was plotted. (D) Mock- and htrb-3 transfected HeLa cells were permeabilised, RNAse treated, and stained with propidium iodide. The DNA content of the cells was measured by flow cytometry.

### Example 4

### Investigation of htrb Cell Cycle Regulation

It has been shown that trb −/− genotype or overexpression of tribbles in Imaginal Disc Cells causes a G2 block and an increase in cell size (Grosshans, J. et al. (2000) Cell 101:523-31; Mata, J. et al. (2000) Cell 101:511-22). This effect was cell-type specific (Grosshans, J. et al. (2000) Cell 101:523-31; Seher, T. C. (2000) Curr. Biol. 10:623-29). As htrbs are the closest known mammalian homologs to tribbles, the effect of htrb expression levels in HeLa cells on the cell size and cell cycle was investigated. As observed with tribbles, elevated or suppressed htrb-3 (and htrb-1, data not shown) levels resulted in an increase in the cell size (FIG. 3C). In contrast, cell cycle distribution was not affected by overexpressed htrb-3 (FIG. 3D). These observations suggest that vertebrate cell size may be controlled by trb proteins, just as Drosophila imaginal disc cell size is under the control of tribbles (Grosshans, J. et al. (2000) Cell 101:523-31; Mata, J. et al. (2000) Cell 101:511-22).

### Example 5

### Analysis of htrb-1 Mediated Repression

Table 1 shows that htrb-3 inhibits AP-1, but not NF-kB, induction by a variety of cytokines. 10 nM final concentration of each cytokine and 50 ng/ml PMA were used to stimulate HeLa cells for 4 hrs. Activation is expressed as relative to the PBS control. Those with the capacity to activate AP-1 reporter were studied for the effect of htrb-3. Results are expressed as percent inhibition caused by the cotransfected htrb-3 construct. Abbreviations: IL- Interleukin, bFGF- Basic Fibroblast Growth Factor, EGF- Epidermal Growth Factor, Ins. like GF- Insulin-like Growth Factor, TGFbeta- Transforming Growth Factor Beta-1, M-CSF- Macrophage Colony Stimulating Factor, G-CSF- Granulocyte Colony Stimulating Factor, GM-CSF- Granulocyte Macrophage Colony Stimulating Factor, PDGF-BB-Platelet-derived Growth Factor-BB, LIF-Leukemia inhibitory Factor, PMA-Tetradecanoylphorbol Acetate.

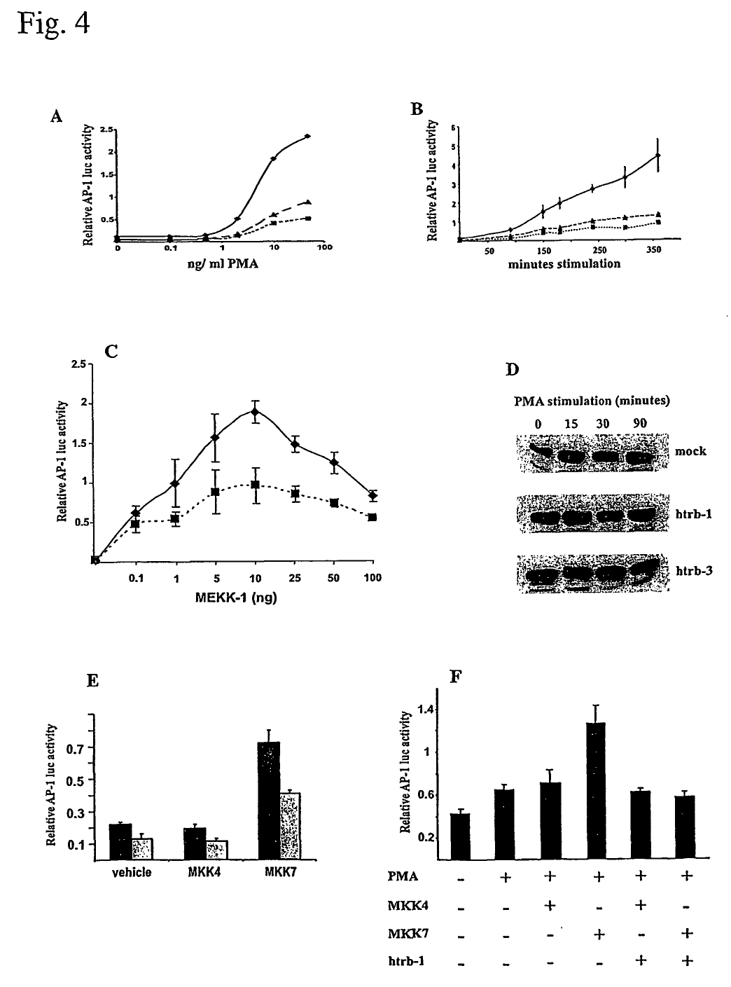

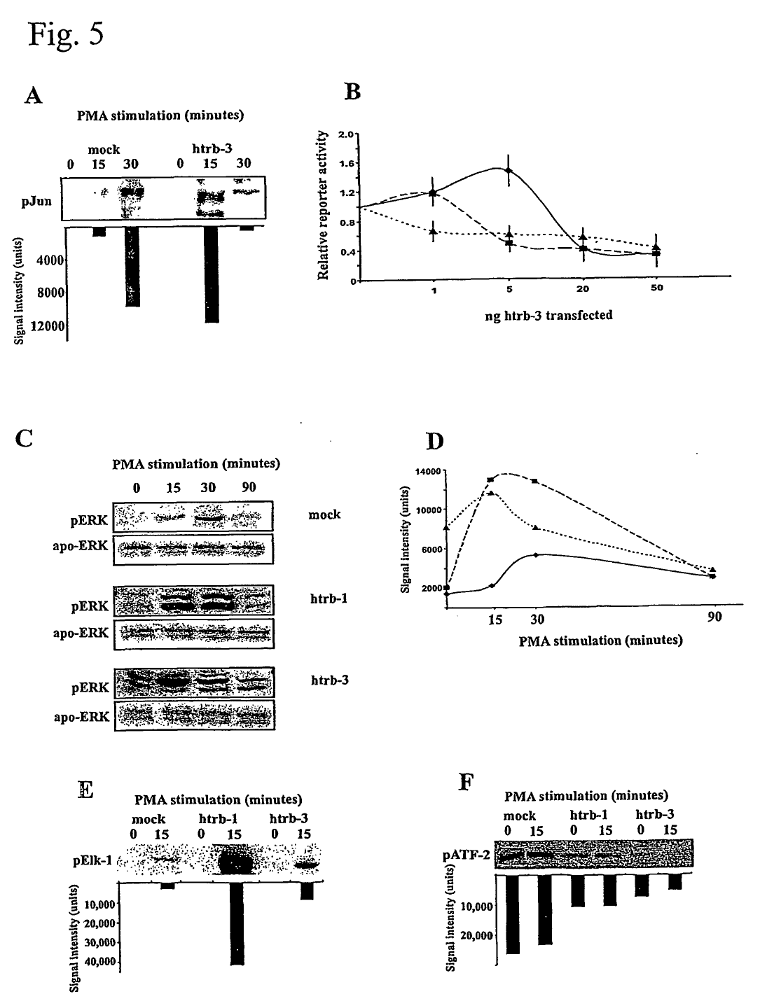

Overexpression of htrb-1 inhibits MEKK-1 mediated AP-1 but not NFkB activation in HeLa cells. A similar effect was seen when the AP-1 reporter was stimulated by PMA (FIG. 4A, B) or by a panel of human cytokines (see Table 1). To characterise the possible site of htrb action, we tested whether increasing the dose of MEKK-1 could bypass the effect of htrb-3 (FIG. 4C). Our data show that this is not the case, suggesting that htrb-3 interacts with a rate-limiting factor downstream of MEKK-1. According to our current understanding, MEKK-1 phosphorylates two downstream kinases, MKK4 (SEKI) and MKK7 (Foltz, I. N. et al. (1998) J. Biol. Chem. 273:9344-51; Holland, P. M. et al. (1997) Journal of Biological Chemistry 272:24994-98). Activation of these kinases leads to phosphorylation of several transcription factors, including c-Jun and CREB2 via Jun kinases (Davis, R. J. (1999) Biochemical Society Symposia 64:1-12). To further characterise the point of action of htrb genes, expression constructs for MKK4 and MKK7 were cotransfected with or without of a htrb-1 expression plasmid (FIG. 4E). MKK7-mediated AP-1 activation was inhibited upon co-expression of htrb-1. Furthermore, activation of AP-1 by PMA was potentiated by overexpressing MKK7 and this activity was blocked upon cotransfection of htrb-1 or htrb-3 (FIG. 4F). The MKK4 construct used did not stimulate the AP-1 reporter (FIG. 4E) nor did it potentiate PMA activation, under the conditions used (FIG. 4F). To examine the possible involvement of MKK4 in htrb action, HeLa cells were transfected with htrb-1 or htrb-3 expression constructs, and stimulated with PMA (FIG. 4D). The phosphorylation level of the endogenous MKK4 protein was not influenced either by PMA or by htrbs. In summary, our data suggest that MKK7 but not MKK4 contributes to AP-1 activation in this system and the inhibitory effect of htrbs is exerted at or downstream of MKK7. To test this, phosphorylation of an effector transcription factor, c-Jun was determined in cell extracts from PMA stimulated HeLa cells (FIG. 5A). While the maximal level of activation was comparable, the kinetics of c-Jun phosphorylation were significantly altered upon htrb-3 overexpression, resulting in a more rapid down-regulation of the stress-kinase response.

FIG. 4 shows that htrb-1 inhibits MEKK-1 and MKK7 mediated AP-1 activation. HeLa cells were transfected with AP-1 reporter (diamonds) and constant doses (50 ng/well) of htrb-1 (triangles) or htrb-3 (squares) expression construct and stimulated with PMA. A Increasing dose of PMA was used to stimulate for 4 hrs. B The effect of 50 ng/ml PMA was followed for 6 hrs. C increasing amount of MEKK-1 expression plasmid (black bars) and constant doses (50 ng/well) of htrb-3 expression construct (gray bars) were cotransfected to study whether overexpression of MEKK-1 can bypass the htrb-3 inhibition. D HeLa cells were transfected with htrb-1 or htrb-3 expression constructs and stimulated 24 hrs post-transfection by 50 ng/ml PMA. pMKK4 levels were followed by western blotting equal amount of total cell lysates. E The activity of AP-1 reporter was measured in response to co-transfection of MKK4 or MKK7 expression constructs in the presence (gray bars) or absence (black bars) of htrb-1 expression plasmid. F HeLa cells transfected with AP-1 reporter, MKK4 or MKK7 expression plasmids +/− htrb-1 expression construct were stimulated with 2ng/ml PMA and the reporter activity was determined.

The overall cellular response to an external MAPK activation stimulus depends on the extent to which individual MAPK pathways are activated. This is a precisely regulated process, which involves many factors, including the micro-environment, cell cycle state, co-stimulatory signals. To investigate whether other MAPK pathways are influenced by overexpression of htrbs, HeLa cells were co-transfected with htrb-3 expression constructs and ERK, JNK or p38 responsive reporters and stimulated by overexpressing upstream MAP kinases, which specifically activate these effector kinases. An increasing dose of htrb-3 was cotransfected (FIG. 5B). As our data demonstrate, a low dose of htrb-3 was able to promote the activation of JNK and ERKs, while p38 activation was suppressed. High htrb doses (>20 ng) inhibited the activation of all MAPK pathways. Furthermore, the optimal htrb-3 dose for facilitating MAPK activation was different for ERKs and JNKs, suggesting that regulation of htrb levels might be a sensitive mechanism to change the balance between the activation of individual MAPK pathways.

### Example 6

### htrb Potentiates ERK Activation

The effect of htrb overexpression on ERK activation was studied. HeLa cells were activated with PMA and the level of phopshorylated ERKs was monitored by western blot (FIG. 5C, D). Our results show that basal level of phospho-ERK was increased by htrb-3 and the maximal stimulation was strongly enhanced as a result of htrb-1 and htrb-3 overexpression. Similarly, increased ERK activity was detected in kinase assays, performed by using PMA activated cell extracts from HeLa cells, where htrb-1 or htrb-3 was overexpressed (FIG. 5E). In contrast to the observed effect of htrb on JNK and ERK phosphorylation levels and kinase activity, p38 kinase activity was suppressed upon overexpressing these proteins (FIG. 5F).

In FIG. 5(A) HeLa cells were transfected with htrb-1 or htrb-3 expression constructs and stimulated by 50 ng/ml PMA. Phospho-cJun levels were determined by western blotting equal amount of total cell lysates and quantitated by NIH Image. (B) pFR luc reporter was transfected into HeLa cells together with pFA-CHOP, activated by pMEK-3 (a p38 activator).(triangles) or with pFA2-Elk-1, activated by pMEK-1 (an ERK activator) (diamonds). AP-1 luc was stimulated by cotransfection with pMEKK-1 to activate the stress kinase cascade (squares). (C) HeLa cells were transfected and treated as on FIG. 5(A). The total and phospho-ERK levels were determined by western blotting. (D) Phospho-ERK levels were quantitated: mock transfected (diamonds), htrb-1 co-transfected (squares), htrb-3 co-transfected (triangles). Kinase assays were performed on PMA stimulated cell extract to measure ERK (E) or p38 (F) activity.

### Example 7

### Analysis of htrb Protein Domain Function

Multiple alignment of trb proteins suggests the existence of three structurally distinct modules (FIG. 2). To investigate the role of these domains in trb function, htrb-1 deletion mutants were generated lacking one or two domains (FIG. 6A). The resulted constructs were expressed HeLa cells as GFP fusion proteins to determine the intracellular localisation of full-length htrb-1 and the deletion mutants, respectively. htrb-1 was found to be localised in the nucleus but excluded from the nucleoli. While deletion of the C-terminal variable domain had no effect on the intracellular distribution (not shown), deleting the N-terminal variable region yielded in a protein which was no longer preferentially localised in the nucleus (FIG. 6B upper panels). Deletion mutants lacking both termini showed a uniform intracellular distribution (not shown). Similar intracellular localisation was found when full length or N-terminal deletion mutant htrb-3-GFP fusion proteins were expressed (FIG. 6B lower panels). The relevance of the different htrb domains in stress kinase signalling was investigated in transient transfection assays. HeLa cells were transfected with the full length and the htrb-1 deletion constructs, with an MEKK-1 expression plasmid as an activator and the effect of htrb mutants on AP-1 activation were determined (FIG. 6C). All htrb-1 deletion mutants showed strong inhibition of MEKK-1 mediated AP-1 activation, suggesting that overexpression of the htrb kinase-like domain is sufficient to inhibit stress kinase signalling. Although the overall protein sequence of htrbs is remarkably similar to tribbles, the proline-rich N-terminal domain, which is responsible for targeting htrb-1 and htrb-3 to the nucleus show the lowest level of homology. This raises the possibility that the mammalian homologs can be targeted to different cellular organelles than the Drosophila protein and/or interact with different proteins.

FIG. 6 shows deletion mutagenesis of htrb-1. (A) trbs share a highly conserved central domain and variable terminal regions. Deletion constructs of htrb-1 were generated lacking one or both variable domains and expressed as a GFP fusion protein. (B) HeLa cells were transfected with expression constructs, expressing the full length or truncated htrb-1 or htrb-3 forms. Truncated constructs lack the N-terminal variable region. 24 hrs after transfection cells were fixed with 5% formaldehyde and counterstained with propidium iodide (PI). Confocal micrographs were taken by exciting PI and GFP. Micrographs were transferred to PowerMacintosh and overlaid. (C) Ability of htrb-1-GFP truncated mutants to inhibit MEKK-1 mediated AP-1 activation was tested in transiently transfected HeLa cells.

We have further shown that an htrb-1 3′UTR construct alone is capable of increasing htrb-1 protein levels, because the steady state levels of expression of endogenous htrb-1 message are increased following overexpression of the htrb-1 3′UTR (data not shown). The mechanism of this regulation may involve a negative regulator of htrb-1 translation that is titrated away in the presence of excess 3′UTR RNA.

### Example 8

### htrb Tissue Specificity of Expression and Cell Specificity of Function

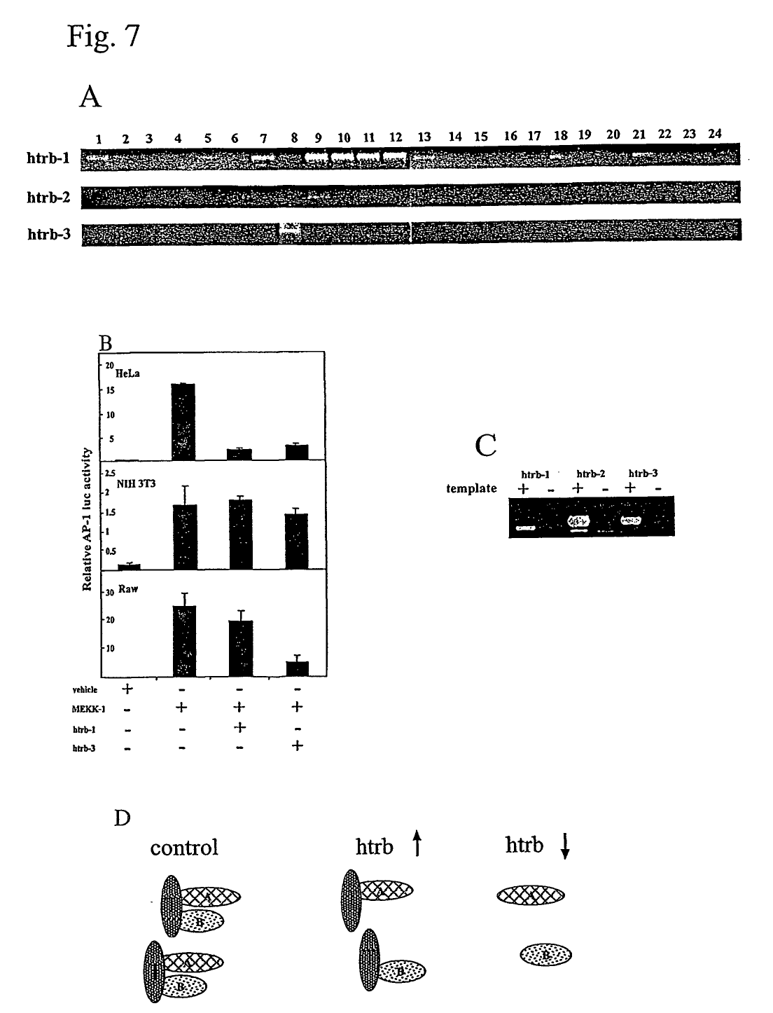

Although htrb proteins show high primary sequence homology to each other, their in vivo function may not be redundant. Specificity is often achieved by tissue specific expression of the individual family members (e.g. JNK or JIP genes). This possibility was tested by determining the mRNA expression profile of all three htrb genes in HeLa cells and quiescent human tissues. HeLa cells express all three htrb genes (FIG. 7C). In contrast, all three htrb genes show restricted expression patterns suggesting tissue specific roles (FIG. 7A). This hypothesis was confirmed by cotransfecting HeLa cells, NIH 3T3 fibroblasts and RAW 264.7 macrophages with htrb-1 or htrb-3 expression constructs and an AP-1 luciferase reporter, and activating them by overexpressing MEKK-1 (FIG. 7B). While both htrb genes disrupted AP-1 activation in HeLa cells, only htrb-3 had an inhibitory effect in RAW cells. Activation of AP-1 in NIH 3T3 cells was not affected by overexpression of either of the htrbs. These results demonstrate that the downstream components of MEKK-1 mediated AP-1 activation and the observed inhibitory effect of htrbs on this signalling cascade are cell type and htrb family member specific.

FIG. 7 shows that htrb genes are expressed and act in a tissue-specific manner. A Multi-tissue PCR was performed to characterise the expression profile of htrb genes by using “Human Rapid-Scan panel” (OriGene) . The following tissues were screened: 1.Brain, 2.Heart, 3.Kidney, 4.Spleen, 5.Liver, 6.Colon, 7.Lung, 8.Small Intestine, 9.Muscle, 10.Stomach, 11.Testis, 12.Placenta, 13.Salivary Gland, 14.Thyroid Gland, 15.Adrenal Gland, 16.Pancreas, 17.Ovary, 18.Uterus, 19.Prostate, 20.Skin, 21.PBL, 22.Bone Marrow, 23.Fetal Brain, 24.Fetal Liver. B HeLa, NIH 3T3 and RAW 264.7 cells were transiently transfected with AP-1 reporter, MEKK-1 expression vector and htrb-1 or htrb-3 expression constructs. C Total RNA was purified from HeLa cells and expression of the htrb genes was tested by RT-PCR. D Working hypothesis for mechanism of the trb action.

### Example 9

### htrb Scaffolding Function

Scaffolding proteins have been demonstrated to be indispensable for the activation of the NFkB and MAPK activation cascades in yeast and mammals (Yamaoka, S. et al. (1998) Cell 93:1231-40; Rudolph, D. et al. (2000) Genes Dev. 14:854-62; Harhaj, E. W. et al. (1999) Journal of Biological Chemistry 274:22911-14; Schaeffer, H. J. et al (1998) Science 281:1668-71; Whitmarsh, A. J. et al. (1998) Science 281:1671-74). Here we describe a family human homologs of Drosophila tribbles. Our data suggest that expression levels of htrb-1 and/or htrb-3 control the amplitude of MAPK activation. According to our hypothesis (FIG. 7D), htrb proteins (“T”) are essential in the assembly of active MAPK complexes (“A”, and “B”). Overexpression or suppression of htrb levels results in the enrichment of inactive complexes and, in turn, reduced MAPK mediated activation. The proposed model of trb action (FIG. 7D), explains both our observations and those in the Drosophila experiments (Grosshans, J. et al. (2000) Cell 101:523-31; Seher, T. C. et al. (2000) Curr. Biol. 10:623-29; Mata, J. et al. (2000) Cell 101:511-22) (FIG. 5B), since scaffolds can facilitate or inhibit signalling responses depending on their concentration (Ferrell, J. E. (2000) www.stke.org/cgi/content/fill/OC_sigtrans;2000/52/pel).

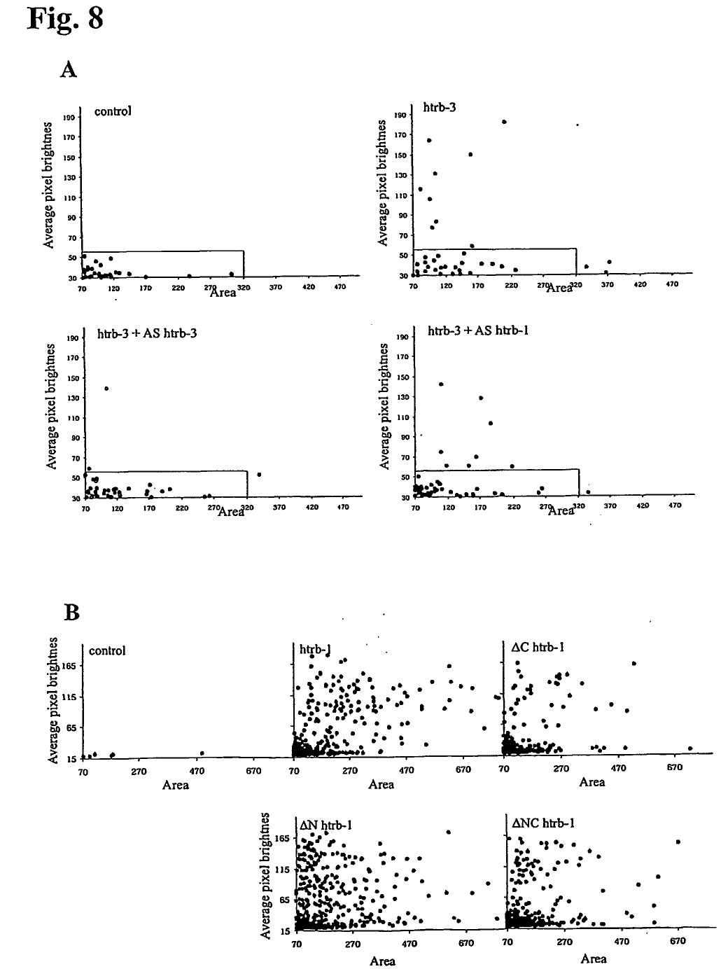

FIG. 8 showsthe detection of htrb-1 and htrb-3 expression by confocal microscopy. A HeLa cells were transiently transfected with htrb-3-GFP expression plasmid, in combination with antisense htrb-3 and antisense htrb-1 expression constructs. 24 hrs later, confocal micrographs were taken using 10× air lenses and htrb-GFP expressing cells were detected by using NIH image (for details of the analysis see: Kiss-Toth, E. et al., Journal of Immunological Methods 239, 125-135 (2000)). The brightness vs. the area of the detected particles is plotted. Red spots indicate cells having the fluorescence above the background level, thus representing htrb-GFP expressors. B HeLa cells were transfected with plasmids expressing full length or truncated htrb-1-GFP proteins. Similarly to panel 8A, expressors were detected on a single cell level.

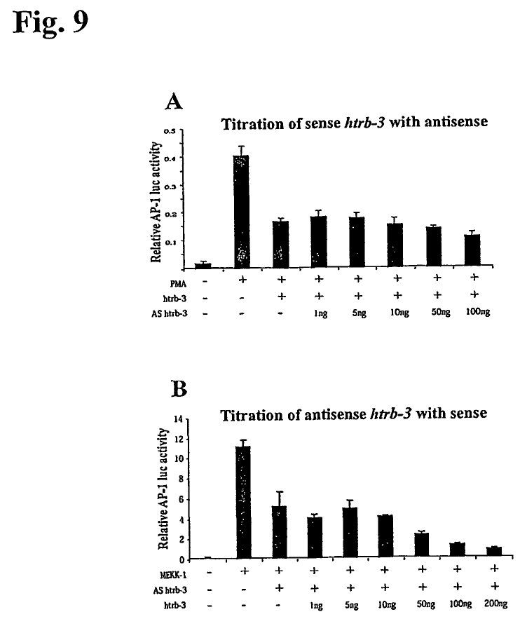

FIG. 9 shows the titration of sense vs. antisense htrb-3. HeLa cells were transfected with the indicated reporter and expression constructs and activated by either PMA (A) or co-transfection of MEKK-1 expression vector (B).

### Example 10

### Materials and Methods

The Human trb sequences reported in this paper have been deposited in the GenBank database: htrb-1 (AF250310), and htrb-3 (AF250311).
SEQ ID No. 1=complete cDNA sequence of htrb-1
SEQ ID No. 2=htrb-1 polypeptide sequence
SEQ ID No. 3=htrb-3 polypeptide sequence
SEQ ID No. 4=htrb-3 polypeptide sequence.

Plasmids, PCR: MKK4, MKK7 (Holland, P. M. et al. (1997) Journal of Biological Chemistry 272:24994-98), IL-8-luc (Wyllie, D. H. et al. (2000) Journal of Immunology 165:7125-32) and LHRE-TK-luc (Maamra, M. et al. (1999) Journal of Biological Chemistry 274:14791-98) were described earlier. V12 Ras was a kind gift of Dr. J. Downward. pAP-1 luc, pNF B luc, pFR luc, pFA-CHOP, pFA2-Elk-1, pMEKK-1 pMEK-1 and pMEK-3 were part of the PathDetect (Stratagene) signal transduction reporter system. htrb clones: Deletion constructs lacking the varible regions were made by PCR using the following primers: htrb-1 5′ deletion primer: 5′-CCGGATCCACCATGATCGCCGACTACCTGCTG-3′, 3′ deletion primer: 5′-CCGGTACCTTACGGCCGAAACCAGGGGTGCAGTAG-3′ htrb-3 5′ deletion primer: 5′-CCGGATCCATCCATCCATGATTGGGCCCTATGTCCTCCTGGAG-3′. PCR products were subcloned into PCR 2.1 TOPO (Invitrogen) and sequenced. For constructing GFP fusion proteins, htrb mutants were subcloned into pEGFPN1 or pEGFPN2 (Clontech), as appropriate.

Cell cultures, transfections: HeLa (ECACC, 85060701) and NIH 3T3 cells were maintained in DMEM with 10% fetal calf serum (FCS) and penicillin-streptomycin. Raw cells were cultured in RPMI supplemented with 10% FCS and penicillin-streptomycin. Cells (1.5×104 per well) were seeded into 96-well tissue culture plates 24 h prior to transfection. Transfections were performed using SuperFect (Qiagen) according to the manufacturer's advice; each well received 500 ng of inducible reporter construct (pIL-8 luc, pAP-1 luc, pNFkB luc orpLHRE-TK luc) 100 ng of pTK-RLuc (Promega) for normalization of transfection efficiency, and 50 ng of htrb-1 or htrb-3 expression vectors under investigation, unless stated otherwise in the appropriate figure legend. 500 ng pFR luc, 100 ng of pTK-RLuc and 10 ng pFA-CHOP or pFA2-ELk-1, 25 ng pMEK-1 or pMEK-3 plasmids were transfected to specifically activate p38 or ERK and to study the effect of htrb-3 on the activation. Sufficient pCDNA3.1 (Invitrogen) (“empty vector”) was added to keep the total DNA dose constant at 700 ng/well. 2 hrs after transfection, cells were washed and 100 ul of fresh medium added. Triplicate wells were transfected for each treatment. Stimulations were performed for 4 hrs (unless indicated otherwise), twenty-four hours later. 2nM IL-1beta or 10 ng/ml TNFalpha, 0.5 ug/ml human growth hormone, 50 ng/ml PMA or 10 nM of the other cytokines, listed on Table 1 was used (unless stated otherwise on the figure). Agonists were prepared and added as 10× stocks in 11 ul of PBS. Reporter levels were measured following 4 hours stimulation using the Dual-Luciferase system (Promega) as recommended by the manufacturer.

Cytokines: IL-1beta was a kind gift from the Immunex Corporation. The other human cytokine preparations were kindly provided by Dr. Steve Poole, NIBSC.

Western blotting: For detection of pJun, pERK and pMKK4, polyclonal antibodies were purchased from Sigma. Protein concentrations of cell lysates were determined and an equal amount of total protein was loaded in each lane. Kinase assays were performed by using the appropriate kits from New England Biolabs.

Equivalents

Those skilled in the art will recognize, or be able to ascertain using no more than routine experimentation, many equivalents of the specific embodiments of the invention described herein. Such equivalents are intended to be encompassed by the following claims.

TABLE 1
A

| | fold AP-1 | fold NFkB |
|---|---|---|
| Cytokine | activation | activation |
| Interferon-α | 3.35 ± 6% | 1.23 ± 13% |
| Interferon-γ | 1.67 ± 5% | 1.25 ± 6% |
| IL-2 | 1.02 ± 17% | 1.00 ± 34% |
| IL-3 | 1.91 ± 7% | 1.22 ± 11% |
| IL-4 | 1.40 ± 26% | 0.95 ± 8% |
| IL-5 | 8.24 ± 8% | 1.74 ± 2% |
| IL-6 | 0.96 ± 11% | 1.05 ± 6% |
| IL-7 | 0.76 ± 24% | 1.10 ± 6% |
| IL-9 | 1.36 ± 16% | 0.86 ± 2% |
| IL-10 | 0.78 ± 5% | 0.89 ± 5% |
| IL-11 | 0.69 ± 8% | 0.76 ± 9% |
| IL-12 | 2.14 ± 11% | 0.69 ± 13% |
| IL-13 | 0.84 ± 10% | 1.08 ± 21% |
| IL-15 | 1.34 ± 10% | 1.76 ± 7% |
| bFGF | 0.72 ± 14% | 1.14 ± 9% |
| EGF | 0.84 ± 4% | 1.28 ± 6% |
| Ins. like GF | 0.69 ± 16% | 1.07 ± 3% |
| TGF-β | 13.37 ± 13% | 6.61 ± 5% |
| Oncostatin M | 0.72 ± 18% | 0.87 ± 6% |
| M-CSF | 1.52 ± 5% | 1.20 ± 16% |
| G-CSF | 0.96 ± 14% | 1.06 ± 15% |
| GM-CSF | 1.04 ± 15% | 1.38 ± 21% |
| PDGF-BB | 9.06 ± 6% | 0.76 ± 18% |
| LIF | 0.73 ± 7% | 0.85 ± 10% |
| Scatter factor | 3.30 ± 8% | 1.03 ± 4% |
| PMA | 10.37 ± 9% | 5.16 ± 15% |

B

| | AP-1 | NFκB |
|---|---|---|
| Cytokine | inhibition | inhibition |
| Scatter factor | 34% | — |
| IL-5 | 46% | −1.5% |
| TGF-β | 30% | 2% |
| Interferon-α | 31% | — |
| IL-12 | 16% | — |
| PDGF-BB | 29% | — |
| PMA | 39% | 0.8% |

1. An isolated htrb-1 encoding nucleic acid comprising a nucleotide sequence which is at least about 90% identical to the nucleotide sequence set forth in SEQ ID No. 1 or the complement thereof.

2. The nucleic acid of claim 1, wherein the nucleic acid comprises a nucleotide sequence at least about 95% identical to the nucleotide sequence set forth in SEQ ID No. 1 or the complement thereof.

3. The nucleic acid of claim 1, wherein the nucleic acid comprises a nucleotide sequence at least about 99% identical to the nucleotide sequence set forth in SEQ ID No. 1 or the complement thereof.

4. The nucleic acid of claim 1, wherein the nucleic acid comprises a nucleotide sequence at least about 95% identical to the nucleotide sequence set forth in SEQ ID No. 1 or the complement thereof, and encodes an AP-1 inhibitory activity.

5. The nucleic acid of claim 1, which hybridize to an htrb-1 ORF encoding nucleic acid corresponding to nucleotides 282 to 1400 of SEQ ID No. 1.

6. The isolated nucleic acid of claim 1, which further encodes an htrb polypeptide that is at least about 75% identical to the htrb-1 polypeptide sequence set forth in SEQ ID No.2.

7. The isolated nucleic acid of claim 6, which further encodes an AP-1 inhibitory activity.

8. The isolated nucleic acid of claim 1, wherein the nucleic acid encodes an htrb bioactivity selected from the group consisting of: an inhibition of IL-8 basal expression, an inhibition of AP-1 transcriptional activation, an inhibition of MEKK-1 kinase signaling, an inhibition of MKK-7 kinase signaling, a cellular hypertrophy-promoting activity, an activation of ERK kinase signaling, and an inhibition of JNK kinase signaling.

9. An isolated nucleic acid comprising a nucleotide sequence that hybridizes under stringent conditions to a htrb-1 nucleotide sequence selected from the group consisting of: nucleotides 283 to 730 of SEQ ID No. 1; nucleotides 1 to 729 of SEQ ID No. 1; and nucleotides 1500 to 1916 of SEQ ID No. 1.

10. The nucleic acid of claim 9, which further encodes an htrb polypeptide that is at least about 50% identical to the htrb-1 polypeptide sequence set forth in SEQ ID No. 2.

11. The nucleic acid of claim 10, which further encodes an htrb-1 bioactivity.

12. The nucleic acid of claim 9, which further includes at least 25 contiguous nucleotides that are identical to said htrb nucleotide sequence.

13. An isolated nucleic acid that encodes the htrb-1 polypeptide sequence set forth in SEQ ID No. 2.

14. An isolated polypeptide comprising a polypeptide sequence of at least 10 contiguous amino acids from the htrb-1 sequence spanning amino acid residues 1 to 150 of SEQ ID No. 2.

15. The polypeptide of claim 14 comprising at least 20 contiguous amino acids from the htrb-1 sequence spanning amino acid residues 1 to 150 of SEQ ID No. 2.

16. An isolated polypeptide comprising an amino acid sequence that is at least 70% identical to the htrb-1 polypeptide sequence set forth in SEQ ID No. 2.

17. The polypeptide of claim 16, wherein the polypeptide sequence is at least 80% identical to the htrb-1 polypeptide sequence set forth in SEQ ID No. 2.

18. The polypeptide of claim 16, wherein the polypeptide sequence is at least 90% identical to the htrb-1 polypeptide sequence set forth in SEQ ID No. 2.

19. The polypeptide of claim 13, having at least one htrb-1 bioactivity.

20. The polypeptide of claim 19, wherein the htrb-1 bioactivity is selected from the group consisting of: an inhibition of IL-8 basal expression, an inhibition of AP-1 transcriptional activation, an inhibition of MEKK-1 kinase signaling, an inhibition of MKK-7 kinase signaling, a cellular hypertrophy-promoting activity, an activation of ERK kinase signaling, and an inhibition of JNK kinase signaling.

21. An isolated htrb-1 polypeptide having the sequence set forth in SEQ ID No. 2.

22. A method of modulating an AP-1 mediated inflammatory signal in a cell comprising providing the cell with a htrb agonist or antagonist.

23. The method of claim 22 wherein the htrb agonist or antagonist is a htrb polypeptide, a htrb peptidomimetic or a htrb nucleic acid.

24. The method of claim 23 wherein the htrb agonist or antagonist is selected from the group consisting of: htrb-1, htrb-1ΔN, htrb-1ΔC, htrb-1ΔNΔC, htrb-3, an htrb-1 5′ UTR and N-terminal variable region antisense construct, an htrb-3 5′ UTR and N-terminal variable region antisense construct, an htrb-1 3′UTR sense construct.

25. The method claim 22, wherein the AP-1 mediated inflammatory signal is selected from the group consisting of: a TNF induced inflammatory signal, and an interleukin induced inflammatory signal.

26. A method of inhibiting an AP-1 mediated inflammatory signal in a cell comprising contacting the cell with an htrb polypeptide of of claim 14.

27. The method of claim 26, wherein the htrb polypeptide is selected from the group consisting of: htrb-1, htrb-1ΔN, htrb-1ΔC, htrb-1ΔNΔC, htrb-3, htrb-3ΔN, htrb-3ΔC, and htrb-3ΔNΔC.

28. A method of activating an ERK-mediated signal in a cell comprising providing the cell with an htrb agonist activity.

29. (canceled)

30. The method of claim 28, wherein the hrtb agonist activity is provided by a htrb polypeptide selected from the group consisting of: htrb-1, htrb-1ΔN, htrb-1ΔC, htrb-1ΔNΔC, htrb-3, htrb-3ΔN, htrb-3ΔC, and htrb-3ΔNΔC.

31. The method of claim 28, wherein the ERK-mediated signal is selected from the group consisting of: an AP-1-mediated gene activation signal, an estrogen receptor-mediated gene activation signal, an FGF induced signal, and a PMA induced signal.

32. A method of identifying an interleukin regulatory gene comprising:
(a) transfecting a mammalian reporter cell comprising an interleukin gene reporter with a low-complexity pool of a mammalian cDNA vector library;
(b) screening the transfected reporter cell for positive clones by identifying transfected cells with either an increase or decrease in the interleukin gene reporter activity relative to the mammalian reporter cell transfected with the vector alone; and
(c) identifying the interleukin regulatory gene from the positive clones by retransfecting the low complexity pool from said positive clones and sequencing the cDNA inserts from the positive clones obtained upon retransfection,
thereby identifying an interleukin regulatory gene.

33. The method of claim 32, wherein the interleukin gene reporter is selected from the group consisting of: an IL-1A gene reporter, an IL-1B gene reporter, an IL-1RN gene reporter, and IL-8 gene reporter.

34. The method of claim 32, wherein the mammalian cell is selected from the group consisting of: a HeLa cell, an NIH 3T3 cell, a Raw cell, a peripheral blood lymphocyte.

35. The method of claim 32, wherein the mammalian cDNA library is selected from the group consisting of: a PBMC library, a HeLa cell library, a PMA-induced mammalian cell library, and a cytokine-induced mammalian cell library.

36. A method of identifying the gene targets of an interleukin regulatory gene in an inflammatory signaling network comprising:
(a) expressing an interleukin regulatory gene clone, comprising an interleukin regulatory gene cDNA and an expression vector, in a population of mammalian cells;
(b) isolating a population of nucleic acids representing expressed genes from said cells;
(c) determining the gene expression profile of the interleukin regulatory gene expressing cells by microarray analysis of the population of nucleic acids representing expressed genes from said cells; and
(d) comparing the gene expression pattern of mRNA expression from the cells transfected with the interleukin regulatory gene clone with that obtained by transfecting the vector alone in order to identify genes, other than the said interleukin regulatory gene, which are either up-regulated or down-regulated in the interleukin regulatory gene expressing cells, thereby identifying the gene targets of an interleukin regulatory gene in an inflammatory signaling network.

37. (canceled)

38. The method of claim 36, wherein the mammalian cell is selected from the group consisting of: a HeLa cell, an NIH 3T3 cell, a Raw cell, a peripheral blood lymphocyte.

39. The method of claim 36, wherein the population of nucleic acids representing expressed genes is an mRNA population.

40. The method of claim 36, wherein the population of nucleic acids representing expressed genes is a cDNA population.

41. The method of claim 36, wherein the microarray analysis provides a gene transcription profile or gene expression fingerprint.


SEQUENCE LISTING (19 sequences)

SEQ ID NO: 1 - DNA, 3319 bp, Homo sapiens
gcggccgcccccagcgaggctccgggagcccttgcctgcgggggtccggggactcgagccggcctccgccccccggacgcacagccagcgtggtccccgcgtgcaacgcgagcgccggggagtggctcctgctttgcccctcgtgggggccgagccaagaccagtctgcaaactccatcccgccggctggaagaagtcgcggagccggcaccaaacccgcagcgtcttcccgcgcggatcccgggacttaaaaagccggggccaccccggcccaggacgggatgcgggtcggtccggtgcgctctgccatgagcggcgcctcgcagccccgcggcccggccctgctcttcccagccacccgaggcgtcccggccaaacgcctgctggacgccgacgacgcggcggctgtggcggccaagtgcccgcgcctctccgagtgctccagccccccggactacctcagcccccccggctcgccctgcagcccgcagcccccgcctgccgctccgggggccggcggaggctccgggagcgcgccggggcccagccgcatcgccgactacctgctgctgcccctagccgagcgcgagcatgtgtcccgggcgctgtgcatccacactggacgcgagctgcgctgcaaggtgtttcccattaaacactaccaggacaaaatcaggccttacatccagctgccatcgcacagcaacattactggcattgtggaagtgatccttggggaaaccaaggcctatgtcttctttgagaagagctttggggacatgcactcctatgtgcgaagccggaagaggctgcgggaagaggaagccgcccggctcttcaagcagattgtctccgccgtcgcccactgccaccagtcagccatcgtgctgggggacctgaagcttaggaagttcgtcttctccacggaggagagaacccagcttagactagaaagtctagaagacacacacataatgaagggggaagatgatgctttgtcagacaaacatggctgcccagcctacgtgagccctgagatcctcaacaccactgggacctactccggaaaggctgcggacgtttggagcctgggggtgatgctctacacccttctggttggacgataccccttccatgactcagaccccagtgcccttttctccaaaattcggcgtggacagttctgcattcctgagcacatttcccccaaagccaggtgcctcattcgcagcctcttgagacgggagccctccgagagactcactgcccccgagatcctactgcacccctggtttgagtccgtcttggaacccgggtacatcgactcagaaataggaacttcagaccagattgttccagagtaccaggaggacagtgacattagttccttcttctgctaatccccaaaacctcagaaacctcataattcttaacacctggcatttccatttctaaagatggacaggccctttggcgtggtaccacccagataatgactgcatcaggatgaaagctgctgacctcggcatggtgcctcctcttctctgggatgagtactttattgatttgagcagcatatctgtgattgctgccctgcaaatttgtttcccttaaggaaccctcaccaactaatctctgctggatttgggagttccgcatcttttgtggagggcagagtatggacatcttacacccggtggtcaagtgtgtaataaacttgagcattcgaatgggagaaaaagcaaatcgcacaatgacatattttgagtaataaccgtatttttcacagggtgacaaattgggccaataaatctgccatctttgaactcatctttggtggctagactgctacggcagcttctctgatgggaaagttccttttttggcttaacactcaccctttcttcacactcacatttaccaatgactctgctccgtttttggagcagactgttttaagttgctcaggagcctgatggaaccatgaaccgagactcttctctgtttcctgccaagacctcatctgcactaatgccttctccctgaccttgacacttccccctttagctataaaagcacttaccagcygaacgtggaacagtatcacaaaagattccatctcccaacgatttcagaactctgagctcagagagactccagattttaaaaaataatttgagtgcttggaaactattagctttttaagttccttccaaatatgttagtacctaccctttactttttccccaagaccatctcagggtggagcattctgtctaagagaagaaagataaggaggctcccacccacctctcccaagagcagacattaaacatctttgtgctttgaagaaagtgaattttggatagtcttgtgattctcagactaacttccagaattatactttaacccctcccagatatggtccgcctttggcattgtgtgtacatctgcagttttgcatggtgggttgttaatatttcaaatgtgtggtttatgaatacgtctgtataatcggcttctggagtgaaacagcaaaccccaaatcttcaaagttggaaggaactttaaaaatcatccggtccaatctctttcctctttctgccacctcccaaggcagaaatcccctcttcagcttcttttgtaggtgggaatccagcctctgttagatatgtccagagatggaaactcactcccctacaaaagatggagcttaatggagaaattgcaactttcattaaaaaacaaattcagatgaaatatcagtaactgtcttggacagtgctgaaatcaggtggttaaacgggtaaacaaaatatactgtattttgagaaatggcacaaaaacaggcagtcatctttaagggctatgcctaggcaaactactaacatgcattgtgagaatgccgtgtatacctcacgtactgtgtactttgtacatatattttaccttttatacctatgttcgattttgttttgttttgttctggctttgaggcttgttttgttgtctgtgtctgtctgaataacctgcgtgtctaaaaccacgtgaaatgtgaatgattattggcaatattaccttgacagaatcatgggactttgagaagagggaggacagaggcctctgtcgcactaacgctctcgtggttgctcgactgttgtatctgtgatacattatccgactaaggactctgggctggcagggccttctgccgggaaagctagaaacactaggttcttcctgtacatacgtgtatatatgtgaacagtgagatggccgtttctgacttgtagagaaattttaataaacctggtttcgt

SEQ ID NO: 2 - protein, 372 aa, Homo sapiens [sequence omitted]

SEQ ID NO: 3 - DNA, 1083 bp, Homo sapiens
atgcgagccacccctctggctgctcctgcgggttccctgtccaggaagaagcggttggagttggatgacaacttagataccgagcgtcccgtccagaaacgagctcgaagtgggccccagcccagactgcccccctgcctgttgcccctgagcccacctactgctccagatcgtgcaactgctgtggccactgcctcccgtcttgggccctatgtcctcctggagcccgaggagggcgggcgggcctaccaggccctgcactgccctacaggcactgagtatacctgcaaggtgtaccccgtccaggaagccctggccgtgctggagccctatgcgcgggtgcccccgcacaagcatgtggctcggcccactgaggtcctggctggtacccagctcctctacgcctttttcactcggacccatggggacatgcacagcctggtgcgaagccgccaccgtatccctgagcctgaggctgccgtgctcttccgccagatggccaccgccctggcgcactgtcaccagcacggtctggtcctgcgtgatctcaagctgtgtcgctttgtcttcgctgaccgtgaccgtgagaagaagaagctggtgctggagaacctggaggactcctgcgtgctgactgggccagatgattccctgtgggacaagcacgcgtgcccagcctacgtgggacctgagatactcagctcacgggcctcatactcgggcaaggcagccgatgtctggagcctgggcgtggcgctcttcaccatgctggccggccactaccccttccaggactcggagcctgtcctgctcttcggcaagatccgccgcggggcctacgccttgcctgcaggcctctcggcccctgcccgctgtctggttcgctgcctccttcgtcgggagccagctgaacggctcacagccacaggcatcctcctgcacccctggctgcgacaggacccgatgcccttagccccaacccgatcccatctctgggaggctgcccaggtggtccctgatggactggggctggacgaagccagggaagaggagggagacagagaagtggttctgtatggctag

SEQ ID NO: 4 - protein, 360 aa, Homo sapiens [sequence omitted]

SEQ ID NO: 5 - protein, 4 aa, Artificial Sequence
  Description of Artificial Sequence Synthetic promoter
LHRE

SEQ ID NO: 6 - DNA, 32 bp, Artificial Sequence
  Description of Artificial Sequence Primer
ccggatccaccatgatcgccgactacctgctg

SEQ ID NO: 7 - DNA, 35 bp, Artificial Sequence
  Description of Artificial Sequence Primer
ccggtaccttacggccgaaaccaggggtgcagtag

SEQ ID NO: 8 - DNA, 43 bp, Artificial Sequence
  Description of Artificial Sequence Primer
ccggatccatccatccatgattgggccctatgtcctcctggag

SEQ ID NO: 9 - protein, 342 aa, Unknown Organism
  Description of Unknown Organism btrb-2 tribble gene sequence
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTNGSYSGKAADVWSLGVMLXXXXXXXXXXXDIEPSSLFSKIRRGQFNIPETLSPKAKCLIRSILRREPSERLTSQEILDHPWFSTDFSVSNSGYGAKEVSDQLVPDVNMEETLDPFF

SEQ ID NO: 10 - protein, 343 aa, Unknown Organism
  Description of Unknown Organism htrb-2 tribble gene sequence
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSAYGAKEVSDQLVPDVNMEENLDPFFN

SEQ ID NO: 11 - protein, 343 aa, Unknown Organism
  Description of Unknown Organism ctrb-2 tribble gene sequence
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSGYGAKEVSDQLVPDVNMEENLDPFFN

SEQ ID NO: 12 - protein, 372 aa, Unknown Organism
  Description of Unknown Organism mtrb-1 tribble gene sequence
MRVGPVRFALSGASQPRGPGLLFPAARGTPAKRLLDTDDAGAVAAKCPRLSECSSPPDYLSPPGSPCSPQPPPSTQGTGGSCVSSPGPSRIADYLLLPLAEREHVSRALCIHTGRELRCKEFPIKHYQDKIRPYIQLPSHSNITGIVEVLLGESKAYVFFEKDFGDMHSYVRSRKRLREEEAARLFKQIVSAVAHCHQSAIVLGDLKLRKFVFSTEERTQLRLGSLEDTHIIKGEDDALSDKHGCPAYVAPEILTTTGTYSGKAADVWSLGVMLYTLWVGRYPFHDSDPSALFSKIRRGQFCIPEHVSPKARCLIRSLLRREPSERLTAPQILLHPWFEYVLEPGYVDSEIGTSDQIVPEYQEDSDISSFFC

SEQ ID NO: 13 - protein, 60 aa, Unknown Organism
  Description of Unknown Organism rtrb-1 tribble gene sequence
MRVGPVRFALSGASQPRGPALLFPAARGTPAKRLLDTDDAAAVAAKCPRLSECSNPPDYL

SEQ ID NO: 14 - protein, 135 aa, Unknown Organism
  Description of Unknown Organism otrb tribble gene sequence
IADYLLLPLAEREHVSRALCIHTGRELRCKVFPIKHYQDKIRPYIQLPSHSNITGIVEVILGEKRAYVFFERSHGDMHSFVRTCKKLREDEAARLFRQIASAVAHCHDNGVVLRDLKLRKFVFRNEDRSLVKLES

SEQ ID NO: 15 - protein, 354 aa, Unknown Organism
  Description of Unknown Organism mtrb-3 tribble gene sequence
MRATPLAASADVSCRKKPLEFDDNIDAECPVLKRVRDEPEPGPLPSLLPPSPPPASDLSPAVAPATRLGPYILLEREQGGRAYRALHCPTGTEYTCKVYPASEAQAVLAPYARLPTHQHVARPTEVLLGSQLLYTFFTRTHGDMHSLVRSRHRIPEPEAAVLFRQMASAVAHCHKHGLVLRDLKLRRFVFSNCERTKLVLENLEDACVMTGSDDSLWDKHACPAYVGPEILSSRASYSGKAADVWSLGVALFTMLAGRYPFQDSEPVLLFGKIRRGAYALPAGLSAPARCLIRCLLRKEPSERLVALGILLHPWLREDHGRVSPPQSDRREMDQVVPDGPQLEEAEEGEVGLYG

SEQ ID NO: 16 - protein, 349 aa, Unknown Organism
  Description of Unknown Organism rtrb-3 tribble gene sequence
MRATSLAASADVPCRKKPLEFDDNIDVECPVLKRVRDEPEPGPTPSLPPASDLSPAVAPATRLGPYILLEREQGNCTYRALHCPTGTEYTCKVYPASEAQAVLAPYARLPTHQHVARPTEVLLGSQLLYTFFTKTHGDLHSLVRSRRGIPEPEAAALFRQMASAVAHCHKHGLILRDLKLRRFVFSNCERTKLVLENLEDACVMTGPDDSLWDKHACPAYVGPEILSSRPSYSGRAADVWSLGVALFTMLAGRYPFQDSEPALLFGKIRRGTFALPEGLSASARCLIRCLLRREPSERLVALGILLHPWLREDCSQVSPPRSDRREMDQVVPDGPQLEEAEEGEVGLYG

SEQ ID NO: 17 - protein, 402 aa, Unknown Organism
  Description of Unknown Organism trb tribble gene sequence
MSSSQEDTVLGLFTPKKEFPNAKMLQTIREKLMTPGGACDLLALGIAAEPTDQQPVKLIQQRYLISAQPSHISAAVAAKTPASYRHLVDLTASNLRCVDIFTGEQFLCRIVNEPLHKVQRAYFQLQQHDEELRRSTIYGHPLIRPVHDIIPLTKDRTYILIAPVPQERDSTGGVTGVYENLHTYIRHAKRLCETETKLQYESLEGSMILDGEDDTLSDKIGCPLYTAPELLCPQQTYKGKPADMWSLGVILYTMLVGQYPFYEKANCNLITVIRHGNVQIPLTLSKSVRWLLLSLLRKDYTERMTASHIFLTPWLREQRPFHMYLPVDVEVAEDWSDAEEDEGTAADAMDDDEEGLCPLGDKHEYEDIGVEPLDYTRSTLQMAQNANGLSTEPEPDTDVDMG

SEQ ID NO: 18 - protein, 181 aa, Unknown Organism
  Description of Unknown Organism atrb tribble gene sequence
SVASGAGSTALYPQRDAISTNVAPPLNAAVLADRYLLLDSVEGSSLYRCVDIKTQEELVCKIANNPCSNLLTAHFRLDGHPHVNCLHKVIPGNNQTYLFFAPSQGDLHSHVRVRKRLREPEARRLFRQMCEVVKTCHEQGIVLRDLKLRKFVFADSERSHLKLESLRGRPLCWTTPAEDLL

SEQ ID NO: 19 - protein, 180 aa, Unknown Organism
  Description of Unknown Organism xtrb-2 tribble gene sequence
EDCHDGGVVLRDLKLRKFVFNDGERTKVKLESLEDAYVLAGSDDSLSDKHGCPAYVSPEILNTNGSYSGKAADVWSLGVMLYTMLVGRYPFHDIEPSSLFSKIRRGQFNIPETLSPKAKCLIRSILRREPSERLTSQEILDHPWFSTDFNALNSGCGAKEVSDQLVPDVNMDEDTDPFFN